(19) United States
(12) Patent Application Publication
(10) Pub. No.: US 2022/0160669 A1
(43) Pub. Date: May 26, 2022

(54) PARENTERAL NUTRITION FORMULATION

(71) Applicants: BAXTER INTERNATIONAL INC., Deerfield, IL (US); BAXTER HEALTHCARE SA, Glattpark (opfikon) (CH)

(72) Inventors: Laurent Christian JEANNIN, I, Braine l'Alleud (BE); Mary HISE BROWN, Vernon Hills, IL (US); Julien A. R. HECQ, Uccle (BE); Gary P. ZALOGA, Evanston, IL (US); Kelly A. TAPPENDEN, Decatur, GA (US)

(21) Appl. No.: 17/103,357

(22) Filed: Nov. 24, 2020

(51) Int. Cl. A61K 31/198; A61K 9/00; A61K 9/08; A61K 45/06

(52) U.S. Cl. CPC A61K 31/198; A61K 45/06; A61K 9/08; A61K 9/0029

(57) ABSTRACT

A composition for use in the treatment of a patient suffering from or being at risk of developing a gastrointestinal disorder is disclosed. The composition is an aqueous solution for injection comprising arginine butyrate in a concentration of from 150 mg/L to 5500 mg/L of the composition. A method of treating a patient suffering from or being at risk of developing a gastrointestinal disorder by administering the related compositions is also disclosed.

### TECHNICAL FIELD

The disclosure is directed to a composition comprising arginine butyrate for use in the treatment or prevention of gastrointestinal disorder in patients suffering from or being at risk of developing such disorder, specifically of pediatric or adult patients receiving parenteral nutrition.

### DESCRIPTION OF THE RELATED ART

Parenteral nutrition (PN) prevents progressive malnutrition and provides lifesaving therapy for many patients with gastrointestinal disorders. However, PN seems to be associated with an increased incidence of infection and inflammation, both local and systemic, in critically ill patients on prolonged parenteral nutrition, where no oral or enteral uptake of any nutrition is possible. Studies have also suggested that impairment of intestinal barrier function might be at least partially responsible (Fukatsu and Kudsk, Surg Clin North Am. 2011; 91(4): 755-770). Accordingly, the intestinal tract's barrier (“intestinal barrier”, “gut barrier” or simply “barrier”, as interchangeably used herein), local and systemic inflammation and local and systemic (nonspecific) immunity have been the object of investigation for many years.

The intestinal tract is lined by a single layer of columnar epithelial cells that forms said gut barrier which allows for selective absorption of nutrients, while restricting access to pathogens and food-borne antigens. Precise regulation of epithelial barrier function is therefore required for maintaining mucosal homeostasis and depends, in part, on barrier-forming elements within the epithelium and a balance between pro- and anti-inflammatory factors in the mucosa. Pathologic states, such as inflammatory bowel disease, are associated with a leaky epithelial barrier, resulting in excessive exposure to microbial antigens, recruitment of leukocytes, release of soluble mediators, and ultimately mucosal damage. An inflammatory microenvironment (herein referred to as “local inflammation”) affects epithelial barrier properties and mucosal homeostasis by altering the structure and function of epithelial intercellular junctions through direct and indirect mechanisms (Luissint et al., Inflammation and the Intestinal Barrier: Leukocyte-Epithelial Cell Interactions, Cell Junction Remodeling, and Mucosal Repair. Gastroenterology 2016; 151(4):616-632) or Alverdy et al.: Total parenteral nutrition promotes bacterial translocation from the gut. Surgery 1988; 104: 185-190.

Gut barrier functionality is also described in Assimakopoulos et al., The Role of the Gut Barrier Function in Health and Disease. Gastroenterology Res 2018; 11(4):261-263. Loss of gut barrier functionality specifically in surgical patients subjected to major operations for various reasons (major liver resection, bowel resection, bowel transplantation etc.) is associated with increased bacterial translocation, i.e. the passage of viable bacteria from the gastrointestinal tract through the epithelial mucosa into the lamina propria and then to the mesenteric lymph nodes and possibly to normally sterile organs, which is in turn associated with infectious complications and promotion of a systemic inflammatory response. An intestinal proinflammatory response further aggravates intestinal injury and danger-associated molecular patterns (DAMPs) are released in the mesenteric lymphatics, carried to the lung and the systemic circulation, stimulating Toll like receptors-4 and potentially other pattern recognition receptors (PRR), thus eventually promoting deleterious effects in diverse organs. Therefore, the gut becomes a proinflammatory organ promoting dangerous effects in even distant organs, through release of DAMPs, even without the need of systemic bacterial translocation. This dramatic effect of gut barrier functionality reduction is extremely relevant for critically ill patients, who are severely injured and/or septic and hospitalized in intensive care units.

Another important aspect of the defense built up by the gut relates to the immune system. Notably, the mucosal immune system provides for about 50%-60% of the body's total immunity, producing about 7% of the antibody made by the human body. For example, it produces specific antibodies against intraluminal bacteria in the form of secretory IgA (sIgA), which does not function through inflammation, but rather through adhesion and bacterial exclusion. In the context of the present invention, this is referred to as the “local immunity” of the gut. The protective role of secretory IgA has generally been evaluated in the context of mucosal infections, where it was shown that IgA acts as a first line of defense by preventing attachment and limiting the access of microorganisms to or beneath the epithelium, a process known as immune exclusion. However, IgA also seems to play a crucial role in maintaining the complex interplay between commensals, epithelium, and immune system (Kato et al. Immunological Reviews 2014; 260: 76-75).

In addition to critically ill patients, barrier dysfunction also involves stable patients with chronic pathologic conditions that promote a chronic immune activation associated with disease progression and/or development of complications and comorbidities from other organs. This intestinal barrier dysfunction group encompasses, for example, patients with HIV infection, liver cirrhosis, chronic viral hepatitis B or C, non-alcoholic steatohepatitis or non-alcoholic fatty liver disease, patients with inflammatory bowel diseases including ulcerative colitis and Crohn's disease, celiac disease, irritable bowel syndrome, obesity and diverse autoimmune conditions (Assimakopoulos et al., The Role of the Gut Barrier Function in Health and Disease. Gastroenterology Res 2018; 11(4):261-263).

While short pauses in oral intake result in minimal alterations in the mucosa/microbial interface, critical illness, with its attendant acidosis, prolonged GI tract starvation, exogenous antibiotics, and breakdown in mucosal defenses may render the host increasingly vulnerable to bacterial challenge. Therefore, a lot of work has already been done to evaluate new chemical entities and their potential role in the development of improved parenteral nutrition formulations which avoid or reduce the effects of long-term PN on the gut as described above, wherein the expression “long-term PN”, as used herein, refers to total parenteral nutrition for more than 7, especially more than 10 days and receive from about 95%-100% of their energy needs from parenteral nutrition, and wherein “total parenteral nutrition” (TPN) means that parenteral nutrition is the only source of nutrition the patient is receiving.

It is known also that the route of nutrition affects the inflammatory response generated by both the innate and the adaptive immunity. It was found that enterally fed animals do have increased levels of intestinal IgA, which may serve to neutralize bacteria within the lumen. As mentioned above, gut starvation with parenteral feeding did not increase intestinal (or lung) IgA, denoting a lack in both innate and acquired mucosal immunity.

The above described issues are relevant for all patients receiving PN, including pediatric and adult patients. For example, pre-term infants, due to transient gut immaturity, often require parenteral nutrition for their first few weeks of life. Children suffering from intestinal failure (IF), for example due to SBS, may even require long-term parenteral nutrition. In addition to the gastrointestinal disorders associated with long-term PN, providing enough protein and energy to sustain their growth and neurodevelopment is a challenge. It could be shown in the past that early parenteral nutrition (PN), including >2.5 g/kg/d of amino acids and at least 40 kcal/kg/d of energy from the first day of life can provide sufficient nutritional intakes for reducing nutritional deficits and the incidence of postnatal growth restriction in preterm infants (Rigo and Senterre, The Journal of Nutrition 143(12), 2013, 2066S-2070S).

Accordingly, it is highly relevant to understand how parenteral nutrition influences the intestinal barrier function, immune cells and inflammatory mediators, and how the composition of a TPN formulation is able to address gastrointestinal disease. In addition, ways to reduce negative effects of long-term TPN on the barrier function, local and potentially also systemic immunity as well as local and systemic inflammation are required. Accordingly, key structural components of the intestinal barrier, specifically the luminal structures termed villi and crypts which are typical for the small intestine, are investigated. For example, short-chain fatty acids have been investigated for their ability to influence gut barrier architecture and function, and, to some extent, also their relevance for the production of IgA.

Short-chain fatty acids (SCFA) are abundant intraluminal solutes in the large intestine and are the primary energy source for the colonic epithelium. SCFAs are produced by anaerobic fermentation of undigested complex carbohydrates, with acetate, propionate, and butyrate being the most abundant of the SCFAs. Physiologic and clinical studies have shown that SFCAs in general and specifically butyric acid may have trophic effects on both the small and large intestines which may be useful for the prevention and treatment of several acute and chronic conditions, and that IV administration of SCFAs ameliorated mucosal atrophy, and butyric acid-supplemented PN (Bu-PN) both increased intestinal mucosal protein synthesis and stimulated the growth of jejunal and ileal cells in an intestinal resection model (Murakoshi et al., Journal of Parenteral and Enteral Nutrition 2011; 35(4): 465-472). It was found that PN supplemented with butyric acid moderately, but significantly, restored PP (Peyer's patches) lymphocyte numbers, as well as intestinal and bronchoalveolar IgA levels, as compared with standard PN. Villous height and crypt depth in the small intestine were significantly decreased in the standard PN group versus the control group, however Bu-PN seemed to restore intestinal morphology.

Another study compared the effects of sodium acetate, sodium propionate and sodium butyrate on rats, where it was found that both intercaecal and intravenous infusion of said SCFA reduced mucosal atrophy (Koruda et al, Am J Clin Nutr 1990; 51:685-689).

Pratt et al., Short-Chain Fatty Acid-Supplemented Total Parenteral Nutrition Improves Nonspecific Immunity After Intestinal Resection in Rats. Journal of Parenteral and Enteral Nutrition 1996; 20(4):264-271, contemplate that the short-chain fatty acids sodium acetate, sodium propionate and sodium butyrate improve components of nonspecific immune responses and that they may be beneficial in reducing certain aspects of TPN associated immunosuppression after major surgery.

Tappenden et al., Short-Chain Fatty Acid-Supplemented Total Parenteral Nutrition Enhances Functional Adaptation to Intestinal Resection in Rats. Gastroenterology 1997; 112:792-802, also describe that Intravenous SCFAs facilitate intestinal adaptation after resection by increasing basolateral intestinal nutrient transport, and that the addition of SCFAs to current TPN formulations may be warranted to improve functional characteristics of the gastrointestinal tract. Also, in this study, sodium acetate, sodium propionate and sodium butyrate were used in the nutrient solutions.

Milo et al., Effects of Short-Chain Fatty Acid-Supplemented Total Parenteral Nutrition on Intestinal Pro-Inflammatory Cytokine Abundance. Digestive Diseases and Sciences 2002; 47:2049-2055, discuss that the short-chain fatty acids acetate, propionate and butyrate beneficially increase small intestinal abundance of IL-1β and IL-6 during total parenteral nutrition administration, while not affecting systemic production of these cytokines or intestinal inflammation.

Bartholome et al., Supplementation of Total Parenteral Nutrition With Butyrate Acutely Increases Structural Aspects of Intestinal Adaptation After an 80% Jejunoileal Resection in Neonatal Piglets. Journal of Parenteral and Enteral Nutrition 2004; 28(4):210-223 state that administration of TPN supplemented with SCFA (acetic acid, propionic acid and n-butyric acid), or butyrate alone, enhances structural indices of intestinal adaptation in the neonatal piglet after massive small bowel resection by increasing proliferation and decreasing apoptosis.

Jirsova et al., The Effect of Butyrate-Supplemented Parenteral Nutrition on Intestinal Defense Mechanisms and the Parenteral Nutrition-Induced Shift in the Gut Microbiota in the Rat Model. BioMed Research International 2019; 2019:1-14, came to the conclusion that in summary, these findings support the hypothesis that butyrate alleviates the detrimental effect of PN on intestinal permeability via the stimulation of tight junction protein expression.

U.S. Pat. No. 5,919,822 A discloses a method for the use of short chain fatty acids in the form of the free fatty acid, triglyceride, diglyceride, monoglyceride, phospholipid or cholesterol ester in lipids for parenteral or enteral nutrition for the maintenance of gastrointestinal integrity and function of a patient whose gut bacteria flora is jeopardized. Free fatty acids mentioned include acetic acid, propionic acid, butyric acid and caproic acid. It is mentioned there that the composition may support disease resistance and immune competence.

U.S. Pat. No. 7,947,303 B2 discloses the use of butyrate, specifically tributyrin, in enteral formulations for improving digestion and absorption in the intestine and for improving the immune status of a patient.

WO 95/11699 A1 describes certain butyric acid derivatives with the treatment of different diseases. For example, it is suggested to use physiologically stable and safe compounds comprising butyric acid salts, butyric acid derivatives and combinations thereof for the treatment or prophylaxis of gastrointestinal disorders including colitis, inflammatory bowel disease, Crohn's disease, and ulcerative colitis. Specifically, it is proposed to administer such compositions by oral or enema formulations, or by rectal irrigation to maximize their contact with and effectiveness on the gastrointestinal system. Arginine butyrate is also generally mentioned, however not as a component of a parenteral nutrition formulation and not in connection with any of the above conditions of the gut.

US 2010/222271 A1 describes formulations for enteral administration comprising protein, polyunsaturated fatty acids, short-chain fatty acids and glutamine, wherein the short-chain fatty acid is butyrate and wherein the formulation may further comprise arginine. It further discloses a method for promoting gastrointestinal health by enterally administering such formulation to a patient.

WO 2019/211605 A1 discloses parenteral nutrition formulations for neonates, wherein the formulations comprise greater than 12% w/v arginine, and their use in the treatment of hypoargininaemia, hyperammonemia, negative nitrogen balance and the prevention of weight loss. However, arginine butyrate is not mentioned, and the influence of arginine on gastrointestinal disorder alone or in combination with other active ingredients is not discussed, and the influence of arginine on gastrointestinal disorder alone or in combination with other active ingredients is not discussed.

Accordingly, the beneficial effects of SCFA, especially butyric acid derivatives, on gut health are well documented. Studies so far have mostly focused on administering sodium butyrate and, to some extent, tributyrin. Sodium butyrate, however, is not an ideal candidate as the sodium load is inevitably increased to the detriment of the patient. Tributyrin, on the other hand, can be associated only with a lipid emulsion for parenteral nutrition, which may not always be the formulation of choice, especially in cases where peripheral administration is preferred or indicated, such as in very young infants. Currently, no medical product for use in parenteral nutrition or for intravenous administration and comprising a butyric acid derivative is available for the treatment of the above-described conditions, including local (gut) inflammation, a compromised gut functionality and integrity, and a compromised local immunity in the gut, often associated with a compromised local immunity in the lung and a compromised general, systemic immunity of the patient. A composition for enteral administration (Intestamin®, Fresenius Kabi) which comprises tributyrin is, however, available for the dietary management of patients with or at risk of disease-related malnutrition in particular for critically ill with limited enteral tolerance. Accordingly, there is a need to provide a medicament for a safe and effective treatment which is able to maintain or improve intestinal barrier functionality, reduces inflammatory events locally and preferably also systemically, and which can maintain or improve local and preferably also systemic immunity, and which at the same time is stable and safe for central or peripheral administration to adults and especially also to infants.

Arginine butyrate (L-arginine, butanoate (3:4)), the butyric acid salt of the amino acid arginine, has been described in some detail in the prior art. However, it has not been contemplated in connection with intestinal diseases such as discussed above and has not been considered as a supplement or active component for the treatment of patients suffering from the above intestinal diseases. Vianello S, Yu H, Voisin V, et al. Arginine butyrate: a therapeutic candidate for Duchenne muscular dystrophy. FASEB J. 2013; 27(6):2256-2269, have discussed arginine butyrate (AB) as a potential drug to treat Duchenne muscular dystrophy, as it combines two pharmacological activities: nitric oxide pathway activation, and histone deacetylase inhibition. Here, arginine was provided as an aqueous solution, wherein arginine was prepared in water and n-butyric acid was added to provide a 26% solution (1 M arginine/1 M butyrate, pH 7) for continuous-chronic injections, and a 12.5% solution (0.76 M arginine/1 M butyrate, pH 5.5) for intermittent injections. Accordingly, aqueous solutions of arginine butyrate which can be administered, for example, intravenously, are known as such.

The prior art also mentions that in EBV-related lymphomas, arginine butyrate induces EBV thymidine kinase transcription and may act synergistically with the antiviral agent ganciclovir to inhibit cell proliferation and decrease cell viability. In addition, the butyrate moiety inhibits histone deacetylase, which results in hyperacetylation of histones H3 and H4. Acetylated histones have a reduced affinity for chromatin; this reduced histone-chromatin affinity may allow chromosomal unfolding, potentially enhancing the expression of genes related to tumor cell growth arrest and apoptosis.

McMahon et al., A randomized phase II trial of arginine butyrate with standard local therapy in refractory sickle cell leg ulcers. bjh 2010; 151(5):516-524, describe the use of arginine butyrate for the treatment of refractory sickle cell leg ulcers.

It was now found that, surprisingly, arginine butyrate compositions, specifically aqueous solutions comprising arginine butyrate in a concentration of from 150 mg/L to 5500 mg/L, can be stably and safely formulated and used in the treatment of local and systemic inflammation, reduced intestinal barrier functionality, and reduced local and systemic immunity, especially in pediatric or adult patients receiving parenteral nutrition, especially long-term parenteral nutrition for various reasons.

### SUMMARY

The inventors have now found that arginine butyrate is unexpectedly effective in improving the intestinal health of parenteral nutrition patients, such as, for example, maintaining or ameliorating local immunity, reducing local inflammation and maintaining or improving intestinal barrier function (also referred to herein as “gastrointestinal disorder”), such as prevalent in, for example, intestinal failure and specifically in patients suffering from short and very short bowel syndrome. Importantly, it was found that arginine butyrate is superior to the butyric acid derivates known as being beneficial for gut health, such as sodium butyrate and tributyrin. Arginine butyrate (AB) was found to be especially effective in reducing local inflammation as well as increasing local immunity. The results also indicate that AB can further reduce systemic inflammation and increase systemic immunity. AB in addition was found to improve intestinal barrier properties and cellular architecture. At the same time, arginine butyrate was found to be stable and safe when formulated, for example, into an aqueous solution for parenteral or intravenous administration either alone or as part of a compounded formulation or when added, for example, to a pre-mixed parenteral formulation, i.e. the AB composition for use in a treatment according to the invention can be used for compounding and can also be added to a parenteral nutrition product before administration to a patient in need. Alternatively, arginine butyrate can also be provided in lyophilized form for reconstitution immediately before use either as a solution for parenteral/intravenous administration of for addition to a parenteral nutrition product (in compounding or for admixing it with a pre-mixed formulation) which is then provided to a patient suffering from or being at risk of developing gastrointestinal disorder.

In light of the disclosure herein, and without limiting the scope of the invention in any way, in a first aspect of the present invention, which may be combined with any other aspect listed herein unless specified otherwise, a composition comprising arginine butyrate in a concentration of from 1 to 3000 mmol per liter is provided for use in the treatment or prevention of local and systemic inflammation, reduced intestinal barrier functionality, and reduced local and systemic immunity.

According to a second aspect of the present invention, the composition for use in a treatment according to the invention is an aqueous solution of arginine butyrate, which is essentially free from carbohydrates and/or amino acids.

According to a third aspect, the composition for use in a treatment according to the invention can comprise further components such as one or more active pharmaceutical ingredients (API) or excipients. API are biologically/pharmacologically active components of a formulation and generate a desired pharmacological effect. Excipients are ingredients which are pharmaceutically inert and are added to a formulation as a carrier of the API. For example, excipients are used to provide bulkiness to formulations, increase the solubility of the API, facilitate absorption of the API, provide stability of the API in the formulation, provide for a controlled release of the API and/or prevent denaturation of the API.

According to a fourth aspect, the composition for use in a treatment according to the invention can further comprise one or more vitamins, preferably water-soluble vitamins, such as, for example vitamin C (ascorbic acid), vitamin B1 (thiamine), vitamin B2 (riboflavin), vitamin B3 (niacin), vitamin B5 (pantothenic acid), vitamin B6 (pyridoxine), vitamin B12 (cyanocobalamin), biotin and folic acid.

According to a fifth aspect, the composition for use in a treatment according to the invention can further comprise one or more trace elements selected from the group of trace elements consisting of copper, chromium, fluoride, iodine, iron, manganese, molybdenum, selenium and zinc.

According to a sixth aspect of the present invention, the composition for use according to the invention comprises arginine butyrate in a concentration of from 250 g to 5.5 g per liter, from 260 mg to 4.5 g per liter, from 280 mg to 3.5 g per liter, from 300 mg to 2.5 g per liter, from 320 mg to 1.5 g per liter, or from 350 mg to 800 mg per liter.

According to a seventh aspect of the present invention, the pH of the composition for use according to the invention is from 5.5 to 8.0.

According to an eighth aspect of the present invention, arginine butyrate is the sole active pharmaceutical ingredient in the composition for use according to the invention.

According to a ninth aspect of the present invention, The composition for use according to the invention is provided to a pediatric or an adult patient, wherein the respective compositions may be adapted, regarding concentrations and doses, accordingly.

According to a tenth aspect of the present invention, the composition for parenteral administration is provided to an intensive care patient, a critically ill patient, a short bowel patient, an intestinal failure patient, a metabolically stressed patient, an immunodeficient patient, a cancer patient, a cachexia patient, a malnourished patient, or a patient suffering from or being at risk of developing reduced gut barrier, hyperglycemia and/or hypertriglyceridemia, ICU patients for whom enteral nutrition is contraindicated, surgical patients with sustained ileus or sustained nothing by mouth (NPO) status, patients with entero-cutaneous fistulas, preterm infants, extreme short bowel patients and/or other home parenteral nutrition (HPN) patients covering 95-100% of their energy needs from parenteral nutrition. The compositions are especially beneficial, for example, for intensive care patients, critically ill patients, short and extreme short bowel patients, and intestinal failure patients. Critically ill patients as mentioned herein encompass patients suffering from sepsis, ischemia reperfusion, necrotizing fasciitis, radiation enteritis, as well as critically ill patients with enteral feeding intolerance, critically ill patients following shock-resuscitation who are hemodynamically unstable, and critically ill patients with enteral intolerance or hemodynamic instability. The compositions are also beneficial for patients having been submitted to radiation and chemotherapy prior to bone marrow transplantation, for patients undergoing/having undergone GI surgery, preterm infants with enteral feeding intolerance, patients suffering from dysmotility, post-intestinal transplant and BMT (bone marrow transplant) patients, patients having a history of MDRO and/or long-term antibiotic use (e.g. endocarditis and osteomyelitis patients) and radiation enteritis patients. For the avoidance of doubt, intestinal failure patients include patients having developed this condition due to short or extreme short bowel syndrome. Specifically, the composition for parenteral administration is provided to short bowel patients, extreme short bowel patients, intestinal failure patients, dysmotility patients, critically ill patients, inflammatory bowel disease patients, post-intestinal transplant patients and cancer patients. The composition for parenteral administration is further indicated for patients suffering from or being at risk of developing gut motility reduction, and/or patients developing IFALD (intestinal failure-associated liver disease). Patients with patient with burns or trauma may also benefit from being treated with a composition according to the invention. Generally, compositions according to the invention are also indicated for home PN patients, as home PN patients, such as, for example, short bowel patients, will require PN on prolonged basis and may not be able to transition quickly to enteral feeding, including the before discussed side-effects of prolonged PN feeding.

According to an eleventh aspect of the present invention, the composition for use according to the invention is provided to a patient who suffers from or is at risk of developing systemic inflammation and/or local inflammation in the gut.

According to a twelfth aspect of the present invention, the composition for use according to the invention is provided for sustaining or improving local immunity in the gut and/or lung of a patient.

According to a thirteenth aspect of the present invention, the composition for use according to the invention is provided for treating or preventing reduced intestinal gut barrier functionality.

According to a fourteenth aspect of the present invention, the composition for use according to the invention is provided for treating or preventing reduced systemic and/or local immunity in the gut.

According to a fifteenth aspect of the present invention, the composition for use according to the invention is administered to the patient by intravenous, intramuscular or subcutaneous injection, preferably by intravenous injection.

According to a sixteenth aspect of the present invention, the composition for use according to the invention is administered to the patient by adding or admixing it with a parenteral nutrition formulation before administration to the patient.

According to a seventeenth aspect of the invention, the composition for use according to the invention is provided in a lyophilized form for dilution or reconstitution in an appropriate diluent before administration to the patient.

According to an eighteenth aspect of the invention, the composition for use according to the invention is administered to the patient to arrive at an arginine butyrate dose of from 5 mg/kg/day to 10 g/kg/day, preferably from 5 mg/kg/day to 5 g/kg/day.

According to a nineteenth aspect, the patient receives the composition according to the invention as long as his or her condition exists and/or until the patient is on and tolerates full enteral nutrition.

According to a twentieth aspect of the invention, the composition according to the invention can be configured for enteral administration.

### BRIEF DESCRIPTION OF THE DRAWINGS

Understanding that figures depict only certain embodiments of the invention and are not to be considered to be limiting the scope of the present disclosure, the present disclosure is described and explained with additional specificity and detail through the use of the accompanying figures.

FIG. 1 shows the average energy delivered to piglets in Groups A, B, C, D, E or P (see Table 1) in kcal/kg body weight/day over 10 days (D1 through D10). Group E refers to the piglets which were fed with milk replacer ad libitum. Group P refers to the group on standard parenteral nutrition (S-PN), whereas Groups A, B, C and D received SCFA-PN, i.e. parenteral nutrition wherein the composition administered was supplemented with tributyrin at 10 mmol/L (Group A) or 30 mmol/L (Group B), arginine butyrate at 10 mmol/L (Group C) and 1,2-dipalmitoyl-3-butyryl glycerol at 10 mmol/L (Group D). Energy uptake was comparable for all piglets on S-PN or SCFA-PN.

FIG. 2 shows the average protein delivered to piglets in Groups A, B, C, D, E or P (see Table 1) in g protein/kg body weight/day over 10 days (D1 through D10). Group E refers to the piglets which were fed with milk replacer ad libitum. Group P refers to the group on standard parenteral nutrition (S-PN), whereas Groups A, B, C and D received SCFA-PN, i.e. parenteral nutrition wherein the composition administered was supplemented with tributyrin at 10 mmol/L (Group A) or 30 mmol/L (Group B), arginine butyrate at 10 mmol/L (Group C) and 1,2-dipalmitoyl-3-butyryl glycerol at 10 mmol/L (Group D). The amount of protein delivered was comparable for all piglets on S-PN or SCFA-PN.

FIG. 3 shows the average development of body weight in kg over the study period (10 days, starting on D0 which is the day of central catheter placement) of piglets in Groups A, B, C, D, E or P (see Table 1). Group E refers to the piglets which were fed with milk replacer ad libitum. Group P refers to the group on standard parenteral nutrition (S-PN), whereas Groups A, B, C and D received SCFA-PN, i.e. parenteral nutrition wherein the composition administered was supplemented with tributyrin at 10 mmol/L (Group A) or 30 mmol/L (Group B), arginine butyrate at 10 mmol/L (Group C) and 1,2-dipalmitoyl-3-butyryl glycerol at 10 mmol/L (Group D). Body weight developed similarly in all groups.

FIG. 4 shows the average development of abdominal girth in cm/kg body weight over the study period (10 days, D1 through D10) of piglets in Groups A, B, C, D, E or P (see Table 1). Group E refers to the piglets which were fed with milk replacer ad libitum. Group P refers to the group on standard parenteral nutrition (S-PN), whereas Groups A, B, C and D received SCFA-PN, i.e. parenteral nutrition wherein the composition administered was supplemented with tributyrin at 10 mmol/L (Group A) or 30 mmol/L (Group B), arginine butyrate at 10 mmol/L (Group C) and 1,2-dipalmitoyl-3-butyryl glycerol at 10 mmol/L (Group D). Abdominal girth developed similarly in all groups.

FIG. 5 shows the average colonic weight in g/cm colon over the study period (10 days, starting on D0 which is the day of central catheter placement) of piglets in Groups A, B, C, D, E or P (see Table 1). Group E refers to the piglets which were fed with milk replacer ad libitum. Group P refers to the group on standard parenteral nutrition (S-PN), whereas Groups A, B, C and D received SCFA-PN, i.e. parenteral nutrition wherein the composition administered was supplemented with tributyrin at 10 mmol/L (Group A) or 30 mmol/L (Group B), arginine butyrate at 10 mmol/L (Group C) and 1,2-dipalmitoyl-3-butyryl glycerol at 10 mmol/L (Group D). Outcome was evaluated based on grouping information using Fisher's LSD test. Groups that do not share a letter are significantly different.

FIG. 6 shows exemplary examples of sections prepared to assess effects in the intestinal histomorphology of the jejunum (A) and the ileum (B). Sections were stained with hematoxylin and eosin. Sections were used to determine the duodenal villous length and duodenal crypt length (FIG. 7 and FIG. 8)

FIG. 7 shows the average duodenal villous length for the respective Study Groups A, B, C and D (see Table I) as well as Group P which is designated “PN” in this FIG. 7. Group E is not shown. Mean duodenal villous length in Group E was 810p. Based on the analysis of data according to Fisher's LSD test, a significant difference was found for Group PN (S-PN), which results in a significantly reduced villous length, and Groups B (tributyrin supplementation, 30 mmol/L TPN, TB-PN) and Group D (1,2-Dipalmitoyl-3-butyryl glycerol supplementation, 10 mmol/L TPN, DPBG-PN), which show an increased villous length compared to the other study groups and especially compared to the PN (S-PN) group. Groups A and C showed about the same results and are only slightly below the mean values determined for Groups B and D.

FIG. 8 shows the average duodenal crypt length for the respective Study Groups A, B, C and D (see Table I) as well as Group P which is designated “PN” in this FIG. 8. Group E is not shown. Mean duodenal crypt depth in Group E was 152p. Based on the analysis of data according to Fisher's LSD test, a significant difference was found again for Group PN (S-PN), which shows the lowest values for crypt depth. Group A (tributyrin supplementation, 10 mmol/L TPN, TB-PN), Group C (arginine butyrate supplementation, 10 mmol/L TPN, AB-PN) and Group D (DPBG supplementation, 10 mmol/L TPN, DPBG-PN) gave the best results, with Group C being slightly better even than Groups A and D. Groups B (tributyrin supplementation, 30 mmol/L TPN, TB-PN), shows a better crypt depth than S-PN, but is not as good as Groups A, C and D.

FIG. 9 shows the average jejunal villous length for the respective Study Groups A, B, C and D (see Table I) as well as Group P which is designated “PN” in this FIG. 9. Group E is not shown. Mean jejunal villous length in Group E was 152p. Based on the analysis of data according to Fisher's LSD test, a significant difference was found especially for Group C (arginine butyrate supplementation, 10 mmol/L TPN, AB-PN), which gave the best results. Jejunal villous length was relatively low in Group B (TB-PN, 10 mmol/L TPN), whereas Group D(DPBG-PN) gave relatively good results as well.

FIG. 10 shows the average jejunal crypt depth for the respective Study Groups A, B, C and D (see Table I) as well as Group P which is designated “PN” in this FIG. 10. Mean jejunal crypt depth in case of EN is shown by a horizontal column for comparison. Based on the analysis of data according to Fisher's LSD test, a significant difference was again found especially for Group C (arginine butyrate supplementation, 10 mmol/L TPN, AB-PN), which gave the best results. Jejunal crypt depth was lower in Groups A, B, D and lowest for Group P (“PN”).

FIG. 11 shows the average ileal crypt depth for the respective Study Groups A, B, C and D (see Table I) as well as Group P which is designated “PN” in this FIG. 11. Mean jejunal crypt depth in case of EN is 155μ. Based on the analysis of data according to Fisher's LSD test, a significant difference was found for Group A (tributyrin supplementation, 10 mmol/L TPN, TB-PN) and Group D (1,2-Dipalmitoyl 3-butyryl glycerol supplementation, 10 mmol/L TPN, DPBG-PN), with Group C being close behind. Jejunal crypt depth was again lowest in Group P (“PN”).

FIG. 12 shows the average colonic crypt depth for the respective Study Groups A, B, C and D (see Table I) as well as Group P which is designated “PN” in this FIG. 12. Mean jejunal crypt depth in case of EN is 76μ. Based on the analysis of data according to Fisher's LSD test, a significant difference was again found for Group C (arginine butyrate supplementation, 10 mmol/L TPN, AB-PN). Jejunal crypt depth was again lowest in Group P (“PN”).

FIG. 13 shows the average jejunal sIgA concentration for the respective Study Groups A, B, C and D (see Table I) as well as Group P which is designated “PN” in this FIG. 13. Based on the analysis of data according to Fisher's LSD test, a significant difference was again found for Group C (arginine butyrate supplementation, 10 mmol/L TPN, AB-PN), which was even more pronounced than for Group E, and even more pronounced than Group P (“PN”).

FIG. 14 provides for CS response times of the piglets from the respective groups, which is used for the assessment of cognitive function. FIG. 14A refers to the Unconditioned Stimulus (US-CS) response time over five days of the study, FIG. 14B refers to the Conditioned Stimulus (CS) response time over five days of the study. No significant difference in the cognitive function could be observed between the Groups.

FIG. 15 shows serum levels of Il-6 in pg per ml serum found in the respective Study Groups A, B, C, D and E (see Table I) as well as Group P which is designated “PN” in this FIG. 15. It can be seen that Group E is lowest, but that the average Il-6 concentration in Group C is significantly lower than in the standard PN group, and also lower compared to other Study (or intervention) Groups.

FIG. 16 shows serum levels of Ill-beta (“Ill-b”) in pg per ml serum found in the respective Study Groups A, B, C, D and E (see Table I) as well as Group P which is designated “PN” in this FIG. 15. It can be seen that Group E is lowest, but that the average Ill-beta concentration in Group C is again significantly lower than in the standard PN group, and also lower compared to other Study (or intervention) Groups.

FIG. 17 shows serum levels of TNF-alpha (“TNF-α”) in pg per ml serum found in the respective Study Groups A, B, C, D and E (see Table I) as well as Group P which is designated “PN” in this FIG. 15. The TNF-alpha concentration in Study Group C is again lower than in the other Study (or intervention) Groups and result in about the same values as Group E.

FIG. 18 shows serum levels of Il-10 in pg per ml serum found in the respective Study Groups A, B, C, D and E (see Table I) as well as Group P which is designated “PN” in this FIG. 15. The TNF-alpha concentration in Study Group E is again lower than in the other Study (or intervention) Groups. Il-a0 concentration are almost or about as low in Study (or intervention) Group D. Study Groups A, B and C have all Il-10 concentrations which are lower than in the standard PN Group P.

FIG. 19 is a schematic depiction of some features of the small intestine wall, some of which have been investigated also in the context of the present invention. The single-layer epithelium (3), comprising cells (5) which carry microvilli on the lumen side, forms the outer layer of the lumen side of the intestine. The intestine is characterized by protruding villi (1) and by crypts (2). The villi are interlaced with blood vessels (4), which allow for a rapid transport of absorbed products. The lacteals (6) absorb lipids from the intestine to the lymphatic system.

### DETAILED DESCRIPTION

Certain embodiments described herein relate generally to the field of parenteral nutrition and the treatment of conditions connected thereto. More particularly, some embodiments described herein relate to compositions or formulations for use in the treatment of patients suffering from or being at risk of develop reduced gut barrier functionality and/or reduced systemic and/or local immunity, specifically in the gut and potentially also in the lung, wherein the composition comprises arginine butyrate. The expression “composition” as used herein is used interchangeably with the expression “formulation” and “medical product”, if not expressly indicated otherwise. The expression “medical product” as used herein is intended to indicate that the composition or formulation for use according to the invention must be fit for the administration to a human or animal body for the safe an effective treatment of the respective disease.

Related embodiments described herein relate to aqueous solution of arginine butyrate which can be used for treating or preventing said conditions in patients who are receiving parenteral nutrition, especially patients receiving long-term parenteral nutrition, wherein the patients can be pediatric or adult patients which require parenteral nutrition for various reasons and are, therefore, at risk of or have developed the said gastrointestinal disorders.

Accordingly, medical products are provided which comprise arginine butyrate in a concentration of from 1 to 3000 mmol per liter of the composition. For example, such formulations are aqueous solutions which can be administered to the patient directly by intravenous, intramuscular or subcutaneous injection. In a preferred embodiment, the composition for use according to the invention is administered intravenously.

Alternatively, the composition for use according to the invention can be the lyophilized form of the medical product, which can be reconstituted before administration to the patient, either directly or by addition to a parenteral nutrition formulation.

An alternative way to provide the composition to the patient comprises adding and admixing the arginine butyrate containing composition with a PN product before administration to the patient. PN products are often provided in 2- or 3-chamber bags, side-by-side, wherein carbohydrates and amino acids and optionally lipid emulsions can be admixed before administration by breaking non-permanent peel seals between the respective chambers. However, certain PN products can also be provided as mono-chamber bags. Generally, such products have a so-called medical port for adding medication which is then administered to the patient together with the PN formulation. Typically, such additional compounds can be certain vitamins or trace elements which cannot be formulated together with the PN formulations and have to be provided to the patient separately. Like that, the formulation of PN products can be individualized to meet specific requirements or to conveniently administer additional medication. Such medical port can, for example, also be used for adding the composition according to the invention to the parenteral nutrition product. However, electrolytes can also be contained in the parenteral nutrition solutions. Lipids are not always part of a parenteral nutrition product. They can be infused separately, if required.

Accordingly, it is one aspect of the present invention to provide for a medical product for use in the treatment of gastrointestinal disorders such as disclosed herein, wherein the product can be administered intravenously, subcutaneously or by intramuscular injection directly or through its addition to a parenteral nutrition product which in turn can be provided to the patient by central or peripheral administration.

According to one aspect of the invention, the composition comprises arginine butyrate in a concentration of 1 mmol to 3000 mmol per liter of the composition, preferably in an aqueous solution. “Aqueous solution” as used herein refers to a solution in which the main solvent of the active pharmaceutical ingredient(s) is water.

Preferably, the composition for use according to the invention does not contain any essential amounts carbohydrates or amino acids which would be considered as nutrients for administration to a patient. As the medical product of the invention is intended to be used for the treatment of gastrointestinal disorders which may occur in connection with parenteral nutrition of a patient, it is desirable to exclude the presence of any such nutrients from the medical product according to the invention to avoid the additional introduction of nutrients to the patient and to allow the targeted and individualized administration of, optionally, both parenteral nutrition products and of the arginine butyrate composition as prescribed and required. Equally, if the medical product according to the invention is not administered to a patient receiving parenteral nutrition but to a patient suffering from gastrointestinal disorders for other reasons, the administration of carbohydrate and/or amino acid macronutrients may not always be required and/or indicated.

Preferably, the composition of the invention comprises arginine butyrate as the sole active pharmaceutical ingredient.

Arginine butyrate can be present in a composition according to the invention in various concentrations, especially where the arginine butyrate is provided as a solution for addition to a parenteral nutrition product where the final arginine butyrate concentration in the (reconstituted) PN formulation can be adjusted as needed. Generally, a composition for use in a treatment according to the invention comprises arginine butyrate in a concentration of up to about 2.2 M (2.2 M arginine, 2.2 M butyrate) in phosphate buffer which can be used, for example, as stock solution for obtaining injectable solutions after dilution with, for example, a 0.9% NaCl solution.

Compositions for infusion according to the invention generally comprise arginine butyrate in a concentration of from 1 mmol to 3000 mmol per liter, from 5 mmol to 3000 mmol per liter, from 1 mmol to 2500 mmol per liter, from 5 mmol to 1250 mmol per liter, from 5 mmol to 750 mmol per liter, from 5 mmol to 500 mmol per liter, from 10 mmol to 100 mmol per liter, or from 10 mmol to 50 mmol per liter.

The compositions for use in a treatment according to the invention can be provided in various volumes of, for example, from 1 to 1000 ml in appropriate containers, for example in volumes of 5 ml, 10 ml, 25 ml, 50 ml, 100 ml, 250 ml or 500 ml. Such containers can be ampoules, bags, bottles, injection syringes or vials. The containers can be made, for example, from glass (vials, bottles ampoules, syringes) or polymeric materials (bottles, bags). Polymeric materials are generally preferred in the context of the present invention as they cannot break, are collapsible and light. For parenteral preparations, the combination of glass containers and elastomeric closures, usually secured by an aluminum cap, can be used. In case of lyophilized forms of the composition of the invention glass vials are preferably to be used. Typical examples are infusion bottles, injection vials and prefilled syringes. Materials which can be used should generally be inert, should not deform and should not release substances which may affect the active ingredient and/or the patient, should protect against biological contamination, light, oxygen and physical damage.

The composition for use as a treatment of gastrointestinal disorder according to the invention can be administered according to a continuous-chronic protocol, such as, for example, daily for at least as long as the gastrointestinal disorder exists and/or as long as there is a risk that a patient develops such disorder, for example as long as said patient receives parenteral nutrition. For example, such continuous-chronic protocol may include the injection of arginine butyrate at 5 mg/kg/day to 1000 mg/kg/day. However, higher doses can be used where necessary and may go up to about 2000 mg/kg/day in adults. The composition for use as a treatment of gastrointestinal disorder according to the invention can also be administered intermittently, such as, for example, once or in a series of consecutive daily injections every 2nd or 3rd week for an extended time or for a fixed period, for example for about 6 to 8 weeks. If administered intermittently, the protocol may include the injection of higher doses of arginine butyrate compared to the continuous-chronic protocol, such as from 5 mg/kg/day up to 2000 mg/kg/day. In both protocols, the composition can be infused intravenously over several hours per day or can be added, where administered to a patient receiving parenteral nutrition, to the parenteral nutrition product of the patient. In addition, the doses administered to a patient can be varied over time for both protocols. For example, therapy can be started with relatively high doses for the first 1 to 5 days, for example with 2 to 3 g/kg/day in adults and then be gradually lowered and adjusted to 50 mg to 500 mg/kg/day. Alternatively, the protocol may comprise escalating doses starting, for example, with 50 mg/kg/day and ending with 3000 mg/kg/day in adults. For pediatric patients, doses can be adjusted to lower values as disclosed herein.

The steady state concentration during an infusion rate of 2000 mg/kg/day was found to be 15.7±4.8 mg/l in Berkovitch et al., Pharmacokinetics of arginine butyrate in patients with hemoglobinopathy. Environmental Toxicology and Pharmacology (1996) 2: 403-405. Arginine butyrate was also found to be rapidly eliminated with a clearance rate of 93.6±31.9 ml/kg/min (clearance rates of 83±12 ml/kg/min were found for sodium butyrate, in comparison), predominantly suggesting an administration of arginine butyrate by continuous infusion for the treatment of gastrointestinal disorder. Accordingly, addition to a parenteral nutrition formulation which is administered to a patient in need and who suffers from or is at risk of developing gastrointestinal disorder is considered a reasonable way of administering arginine butyrate.

The expression “lyophilized” as used herein refers to a product comprising arginine butyrate which is obtained after lyophilization of a composition according to the invention. The lyophilized product can be dissolved before use in WFI to obtain the reconstituted composition for injection. Alternatively, water can be added by means of a parenteral formulation, such as an aqueous amino acid or an aqueous carbohydrate formulation or a reconstituted formulation from an MCB, wherein the parenteral formulation will generally be drawn from a parenteral nutrition product to which the composition according to the invention is then again added after dilution, for administration of the arginine butyrate spiked parenteral nutrition formulation to a patient in need. Examples for such reconstitution of e.g., lyophilized composition are described in WO 2019/032934 A1, WO 2019/245960 A1 and WO 2020/154292 A1.

The expression “pediatric” as used herein refers to neonates, including premature (pre-term), full term, and post-mature neonates of up to one month of age; infants of between one month and one year of age; children of between one and up to 12 years of age, and adolescents of between 13 and up to 21 years of age. The formulations according to the invention are specifically suitable for neonates, including pre-term, full-term and post-mature neonates. The formulations are especially suitable for pre-term neonates, who may have a birthweight of below 2500 g, of below 2000 g, of below 1800 g, of below 1500 g, of below 1200 g, of below 1100 g, or even of below 1000 g.

The expression “short-chain fatty acid” or “SCFA” as used herein refers to fatty acids with fewer than six carbon atoms. Table 1 provides for a list of the most common short-chain fatty acids, their common and systemic names as well as their formulas.

TABLE 1
List of short-chain fatty acids and their respective
names and formulas

| Lipid | Name | | Salt Name | | Formula | |
|---|---|---|---|---|---|---|
| Number | Common | Systematic | Common | Systematic | Molecular | Structural |
| C1:0 | Formic | Methanoic | Formate | Methanoate | CH₂O₂ | HCOOH |
| | acid | acid | | | | |
| C2:0 | Acetic | Ethanoic | Acetate | Ethanoate | C₂H₄O₂ | CH₃COOH |
| | acid | acid | | | | |
| C3:0 | Propionic | Propanoic | Propionate | Propanoate | C₃H₆O₂ | CH₃CH₂COOH |
| | acid | acid | | | | |
| C4:0 | Butyric | Butanoic | Butyrate | Butanoate | C₄H₈O₂ | CH₃(CH₂)₂ |
| | acid | acid | | | | COOH |
| C4:0 | Isobutyric | 2- | Isobutyrate | 2- | C₄H₈O₂ | (CH₃)₂ |
| | acid | Methyl- | | Methyl- | | CHCOOH |
| | | propanoic | | propanoate | | |
| | | acid | | | | |
| C5:0 | Valeric | Pentanoic | Valerate | Pentanoate | C₅H₁₀O₂ | CH₃(CH₂)₃ |
| | acid | acid | | | | COOH |
| C5:0 | Isovaleric | 3- | Isovalerate | 3- | C₅H₁₀O₂ | (CH₃)₂CHCH₂ |
| | acid | Methyl- | | Methyl- | | COOH |
| | | butanoic | | butanoate | | |
| | | acid | | | | |

Short-chain fatty acids including butyric acid are liquid at room temperature and generally have a pungent or rancid odor, which makes it difficult to use them in therapy. Their alkali metal salts are hydrolyzed in aqueous solutions. They are described in some detail in Schönfeld and Wojtczak, Short- and medium-chain fatty acids in energy metabolism: the cellular perspective. J Lipid Res 2016; 75(6):943-954.

The expression “parenteral nutrition” (PN) as used herein refers to the intravenous administration of nutritional components, which may include protein, carbohydrate, fat, minerals and electrolytes, vitamins and trace elements, to patients who cannot eat or absorb enough food through tube (enteral) feeding to maintain good nutrition status. Diseases and conditions where PN is indicated include but are not limited to short and extreme short bowel syndrome, intestinal failure, GI fistulas, bowel obstruction, patients with radiation and chemotherapy prior to bone marrow transplantation, patients undergoing/having undergone GI surgery, preterm infants with enteral feeding intolerance, critically ill patients, and severe acute pancreatitis. Critically ill patients encompass patients suffering from sepsis, ischemia reperfusion, necrotizing fasciitis, radiation enteritis, as well as critically ill patients with enteral feeding intolerance, and critically ill patients following shock-resuscitation who are hemodynamically unstable. Patients receiving PN as described above include pre-term or newborn babies, infants, children, adolescents and adults.

The expression “parenteral nutrition solution” as used herein generally refers to a sterile liquid chemical formula suitable for parenteral nutrition and which is given directly into the bloodstream of a patient through an intravenous (IV) catheter. A parenteral nutrition solution provided in, for example, a multi-chamber container is considered a medical product.

The expression “total parenteral nutrition (TPN)” as used herein implies that all macronutrient (carbohydrate, nitrogen and lipid) and micronutrient (vitamins, trace elements and minerals) and fluid requirements of a patient are met by an intravenous nutrient solution and no significant nutrition is obtained from other sources.

The expression “gut” as used herein, refers to the intestine. The expressions are interchangeably used herein. The intestine consists of the small intestine, colon (large intestine) and rectum. The small intestine is divided into the duodenum, jejunum, and ileum.

The expression “sole ingredient” as used herein means that no further pharmaceutically active agents that are effective against the condition being treated are present. This includes compounds which are generally provided to a patient receiving parenteral nutrition, including, but not limited to, macro- and micronutrients intended to address the nutritional needs of the patient. However, excipients, including but not limited to diluents such as water, saline solution, polyethylene glycols, glycerine, propylene glycol or other synthetic solvents, antibacterial agents such as, for example, benzyl alcohol or methyl parabens, antioxidants, such as, for example, ascorbic acid or sodium bisulfite, chelating agents such as, for example, EDTA, or buffer such as, for example, acetates, citrates or phosphates or dextrose may be present.

The expression “reconstitution” as used herein, refers to the mixing of fluids or formulations contained in distinct containers or chambers, for example within a multi-chamber bag. In case of a multi-chamber bag, reconstitution is achieved by opening or breaking one or more non-permanent (peel) seals which separate the chambers and the fluids contained therein. A “reconstituted” fluid thus is a fluid which is obtained by mixing two or more fluids or formulations located in different chambers of a multi-chamber bag or, alternatively, in distinct containers. Such reconstitution is generally done shortly before administration of the reconstituted composition to a patient.

The expression “for injection” as used herein refers to the administration of a liquid into the body of a patient, including subcutaneous injection or infusion and intravenous (IV) injection or infusion into a vein. The “infusion” of a solution or formulation refers to the prolonged administration of such solution or formulation in contrast to an injection, which refers to the quick administration (a “shot”) of said formulation or solution. Infusion can be achieved either by adding the solution or composition to a formulation which a patient receives over a prolonged time, such as a parenteral nutrition formulation, for example by means of a medical port as described herein. Alternatively, compositions or formulations as described herein can be administered by “piggyback intravenous infusion”, which is the intermittent delivery of an additional formulation or medication through the primary intravenous line from a second source of fluid with a secondary set of intravenous tubing. Finally, a “push intravenous infusion” is the direct injection of medication into a vein through an intravenous line, needle, or catheter. All the aforementioned methods can be used in the context of the invention. A solution, composition or formulation “for injection” is a solution, composition or formulation which is suitable for injection, including being a sterile, nonpyrogenic preparation which contains no bacteriostat, antimicrobial agent or added buffer, is an approximately isotonic solution and has an appropriate pH.

The expression “gastrointestinal disorder” or “gastrointestinal disorders” as used herein generally refers to disorders of immunological barriers and the inflammatory response mediated by a challenged or reduced gut barrier functionality. Specifically, it refers to (i) intestinal inflammation potentially associated with the upregulation of (pro-)inflammatory mediators and development of systemic inflammation; (ii) reduced local immunity in the gut (intestinal and mucosal immunity) and optionally also in the lung, mediated by reduced secretion of intestinal and respiratory IgA; reduced systemic immunity; (iii) reduced gut barrier functionality, including, but not limited to, degradation of the gut architecture and disruption of mucosal homeostasis associated with increased intestinal permeability, inhibition of vitamin and nutrient transport and a reduction in sodium and water absorption, as well as increased bacterial transfer. Gastrointestinal disorders as described herein are specifically associated with patients receiving parenteral nutrition, especially long-term parenteral nutrition, and/or patients suffering from intestinal failure.

The expression “intestinal inflammation” as used herein refers to a dysregulated mucosal immune response to antigens that is characterized by an excessive proinflammatory cytokine proliferation derived from CD4+ T cells over and above the response that is normally associated with tolerance and immunoregulation derived from T-regulatory cells (Blumberg, Inflammation in the Intestinal Tract: Pathogenesis and Treatment. Digestive Diseases 2009 27(4):455-464).

The expression “systemic inflammation” as used herein refers to inflammation affecting the entire body rather than a single organ or body part, which is referred to herein as “local inflammation. The expression “inflammation” as used herein refers to the response of body tissues to harmful stimuli, including the production of eicosanoids and cytokines.

The expression “systemic immunity” as used herein refers to the state of a human being having adequate biological defenses to fight infection, disease, or other unwanted biological invasion. “Local immunity” or “regional immunity” as used herein refers to the ability of a body part or organ to fight infection, disease, or other unwanted biological invasion. In the context of the present invention, “local immunity” relates to the ability of the intestine and optionally also the lung to effectively respond to such infection, disease, or other unwanted biological invasion.

The expression “intestinal failure” (IF) as used herein refers to the reduction of gut function below the minimum necessary for the absorption of macronutrients and/or water and electrolytes, such that intravenous supplementation is required to maintain health and/or growth”. IF encompasses type I acute intestinal failure (AIF), type II prolonged AIF and type III chronic intestinal failure (CIF), all of which are covered hereunder. The pathophysiological mechanism of CIF includes, for example, short bowel syndrome (SBS) or end jejunostomy, and underlying diseases comprise Crohn's disease, mesenteric ischaemia, surgical complications, primary chronic intestinal pseudoob-struction and radiation enteritis. Underlying diseases for prolonged AIF encompass, for example, surgical complications, Crohn's disease, motility disorders, and intestinal ischaemia and malignancy. Intestinal failure patients also include those not suffering from short bowel syndrome, including dysmotility patients, critically ill patients with enteral intolerance or hemodynamic instability, IBD patients, and post-intestinal transplant patients.

The expression “short bowel syndrome” (SBS) as used herein refers to a malabsorption disorder caused by a lack of functional small intestine. SBS can occur when portions of the small intestine have been surgically removed, for example because of Crohn's disease, cancer, traumatic injuries and blood clots in the arteries that provide blood to the intestines. SBS also occurs when portions of the small intestines are missing or damaged at birth.

In the context of the present invention the expression “patient” refers to a human or animal patient. The human patient can be a pediatric or adult patient. Specifically, the patient is a patient who is receiving parenteral nutrition, including long-term parenteral nutrition or a patient suffering from intestinal failure and/or short or extreme short bowel syndrome.

The expression multi-chamber container(s) (MCB) as used herein refers to nutritional formulations for parenteral administration to a patient which are provided in a container which may be in the form of a bag having multiple compartments or chambers. Such container includes at least two chambers, but can also include three, four, five or more chambers. Suitable containers, including bags, typically are sterile, nonpyrogenic, single-use, and/or ready-to-use products. The multi-chamber containers are particularly useful for holding a parenteral nutrition product and typically provide a carbohydrate formulation, an amino acid formulation, and optionally a lipid formulation as disclosed herein in a third chamber of the container. The multi-chamber containers may also provide a fourth chamber or a fifth chamber which comprise, for example, certain drugs, vitamins and/or trace elements which cannot be admixed to the carbohydrate, amino acid or lipid formulation or the addition of which is intended to be optional.

The expression “peripheral parenteral nutrition (PPN)” as used herein refers to the administration of PN solution via a cannula inserted into a peripheral vein. The term “peripheral” refers to superficial veins, most often of the upper extremities. PPN is indicated, for example, for short-term PN, when catheterization of a central vein is not required, contraindicated or impossible, such as in case of catheter sepsis or bacteremia. In contrast, “central parenteral nutrition” refers to parenteral nutrition which is given via a central vein and catheter. Central access allows for the administration of highly concentrated, hypertonic solutions, and are often used for patients requiring PN for more than 2 weeks. Either a temporary central venous catheter (CVC) or longterm CVC, such as a tunneled catheter, an implanted port, or a peripherally inserted central catheter (PICC) can be used. As CVCs can increase catheter-related blood stream infections, peripheral parenteral nutrition (PPN) is used where indicated and possible.

According to another aspect of the invention, the composition for use in the treatment of gastrointestinal disorders may additionally comprise further active pharmaceutical ingredients.

Isotonic agents can also be added to the composition of the invention to adjust the osmolarity to a desired level, such as a physiologically acceptable level. Suitable isotonic agents include, but are not limited to, glycerol, propylene glycol, sorbitol, mannitol, dextrose, lactose or sodium chloride. In some cases, the composition of the invention includes an isotonic agent in an amount of about 1% to about 10% by weight based on the total weight of the lipid formulation, such as about 1% to about 5%, about 1% to about 4%, and/or about 2% to about 3%. In some cases, the lipid emulsion formulation includes about 2% to about 3% by weight of glycerol.

pH modifiers can be added to the composition to adjust the pH to a desired level, such as a physiologically acceptable pH for intravenous use. Suitable pH modifiers include, but are not limited to, sodium hydroxide and hydrochloric acid. Typically, the composition of the invention has a pH of about 6 to about 9, such as about 6.1 to about 8.9, about 6.2 to about 8.8, about 6.3 to about 8.7, about 6.4 to about 8.6, about 6.5 to about 8.5, about 6.6 to about 8.4, about 6.7 to about 8.3, about 6.8 to about 8.2, about 6.9 to about 8.1, about 7 to about 8, about 7.1 to about 7.9, about 7.2 to about 7.8, about 7.3 to about 7.7, about 7.4 to about 7.6, about 7, about 7.5, and/or about 8.

The water used for preparing the composition according to the invention, including reconstitution of a lyophilized form of the product, must conform to the pharmacopeial requirements that make it suitable for injection, that is the water must be sterile water for injection (WFI).

The arginine butyrate supplemented parenteral nutrition formulations can be prepared, for example, by providing a lyophilized composition thereof or a concentrated or stock solution comprising arginine butyrate according to the invention, and by dissolving or optionally diluting said composition in appropriate solutions, including, for example, isotonic (normal) saline, lactated Ringer, dextrose %5 in water or potassium chloride 0.2% in 5% dextrose. From the resulting solution an amount required for generating the desired final concentration in the administered product is added to the parenteral nutrition formulation. Alternatively, compositions for a treatment according to the invention can be administered by means of “piggyback intravenous infusion”, an intermittent delivery of an additional formulation or medication through the primary intravenous line (e.g. for PN) from a second source of fluid with a secondary set of intravenous tubing (the composition according to the invention). Finally, a “push intravenous infusion” can be used. The composition for use in a treatment according to the invention can also be formulated to be directly injected subcutaneously or intravenously on a regular or intermittent basis.

Compositions according to the invention can be prepared by filling a cleaned and nitrogen flushed mixing tank with a first batch of water for injection. When the required temperature is reached, optionally further components as disclosed herein and arginine butyrate are added together or sequentially to the tank. Agitation is initiated, and the solution is adjusted to the final volume with water for injection. The pH of the solution is measured and if needed adjusted to the required pH. The solution is visually checked to ensure it is a clear solution.

During the filling process, the aqueous arginine butyrate solution is filtered through a 0.45 μm filtration membrane. The fill volume is determined gravimetrically and is periodically checked during the filling process to ensure uniformity across the batch. Additionally, dissolved oxygen can be measured and adapted, where necessary, on the first filled containers. The containers are then sealed. The containers are placed on sterilizer trays for moist heat sterilization. The product can be terminally sterilized at 121° C. and 2.2 bar using a moist heat sterilization process adapted for the selected sizes/volumes of the containers. For example, a Steam-Air Mixture process can be utilized. The exposure time is adapted to the size/volume of the container.

The arginine butyrate was found to be stable during production, sterilization and over a shelf-life of at least 12 months at 25° C. and 40% relative humidity (RH) in an amino acid formulation (see also Example 1 and Table 5). An aqueous arginine butyrate composition according to the invention was found to be equally producible and stable in concentration of up to 5500 mg/L (see Example 4. According to one aspect of the invention, it may be beneficial to provide arginine butyrate solutions according to the invention which have a higher arginine butyrate concentration, as low volumes can then be used in a treatment according to the inventions. Concentrations contemplated herein comprise arginine butyrate solutions of up to 25% (w/v), of up to 21% (w/v), 15% (w/v) or 10% (w/v).

The disclosure also provides methods of treating patients who suffer from or are at risk of developing a gastrointestinal disorder. According to one aspect of the invention, the treatment according to the invention is directed at patients who receive parenteral nutrition when oral and enteral nutrition is not possible, insufficient or contraindicated. According to another aspect of the invention, the treatment is especially directed at patients suffering from intestinal failure and/or short or extreme short bowel syndrome, including, but not limited to patients who receive parenteral nutrition due to their condition. Such patients may include ICU patients, critically ill patients or hospitalized patients, but also home patients, especially those patients mentioned who cover more than 50%, preferably more than 60%, 70%, 80% or 90% of their energy needs from parenteral nutrition The methods involve administering a composition according to the invention by injection either as disclosed herein.

In pediatric patients, the formulations according to the present invention are administered in a way to arrive at an arginine butyrate dose of from 5 mg/kg/day to 5 g/kg/day, preferably from 5 mg/kg/day to 4 g/kg/day, or from 5 mg/kg/day to 2 g/kg/day. The dose may have to be adapted depending on the age of the pediatric patient and/or on nutrient uptake via other than the parenteral route. For example, the dose may have to be adapted or reduced if the pediatric patient additionally receives enteral nutrition.

It is to be understood that the initial doses of arginine butyrate administered to a patient may be low (e.g. 5 mg/kg/day) and may gradually be increased (to e.g. 100 mg/kg/day, 1 g/kg/day, 2 g/kg/day, 3 g/kg/day, 4 g/kg/day, or 5 g/kg/day) and that the doses may have to be adapted depending on the patient's status. Where the composition of the invention is administered by addition to another fluid, such as a parenteral nutrition solution or an IV solution, directly or by piggyback intravenous infusion, doses can be managed, for example, by adjusting the added arginine butyrate composition to the flow rate with which the ON or IV solution is administered, or by adjusting the flow rate to the intended daily dose of arginine butyrate. For example, a PN product such as NUMETA G13E can be administered with flow rates as high as 127.9 ml/kg/day. It is known that, for example, arginine butyrate, as an aqueous solution, pH 7.7, was tolerated in a dose of 3 g/kg/day during a phase I clinical trial addressing metastatic colorectal cancer patients (Douillard et al., Phase I trial of interleukin-2 and high-dose arginine butyrate in metastatic colorectal cancer. Cancer Immunology Immunotherapy 2000; 49:56-61).

In adult patients, the formulations according to the present invention are administered in a way to arrive at an arginine butyrate dose of from 5 mg/kg/day to 10 g/kg/day, preferably from 5 mg/kg/day to 5 g/kg/day, or from 500 mg/kg/day to 4 g/kg/day, or from 1 g/kg/day to 4 g/kg/day. The dose may have to be adapted depending on nutrient uptake via other than the parenteral route, e.g. if the patient additionally receives enteral nutrition. Required doses can be administered, for example, by adjusting the flow rate with which PN is administered.

Preferably, the maximum dose of arginine butyrate is 4 g/kg/d, and especially preferably 3 g/kg/d.

The small intestine has three different regions: the duodenum, jejunum, and ileum. The duodenum, the shortest, is where the absorption of compounds through small finger-like protrusions called villi is prepared and where it starts. The jejunum is specialized on absorption through its lining by enterocytes: small nutrient particles which have been previously digested by enzymes in the duodenum are taken up. The main function of the ileum is to absorb compounds such as vitamin B12, bile salts, and other products of digestion which were not absorbed by the jejunum. Villi are projections into the lumen covered predominantly with the above mentioned mature, absorptive enterocytes, along with occasional mucus-secreting goblet cells. Each villus is approximately 0.5-1.6 mm in length (in humans) and has many microvilli projecting from the enterocytes of its epithelium which collectively form the striated or brush border. Each of these microvilli are much smaller than a single villus. The intestinal villi are again much smaller than any of the circular folds in the intestine. Crypts are moat-like invaginations of the epithelium around the villi and are lined largely with younger epithelial cells which are involved primarily in secretion. Importantly, toward the base of the crypts are stem cells, which continually divide and provide the source of all the epithelial cells in the crypts and on the villi.

Healthy villi and crypts, together with their cell lining (FIG. 19), are an important marker for a functional small intestine. Villi increase the internal surface area of the intestinal walls for efficient absorption. An increased absorptive area is useful because digested nutrients (including, for example, amino acids) pass into the semipermeable villi through diffusion, which is effective only at short distances. In other words, increased surface area (in contact with the fluid in the lumen) decreases the average distance travelled by nutrient molecules and in turn increases the effectiveness of diffusion and nutrient uptake. The villi are connected to blood vessels, whereby nutrients can be transported away. Atrophied villi tend to be shorter and crypts tend to be less pronounced, with a lower depth. Accordingly, assessing the length of villi and the depth of crypts in the various sections of the small intestine, i.e. the duodenum, jejunum and the ileum, provide for a relevant information on the health of the small intestine (Burrin et al., Translational Advances in Pediatric Nutrition and Gastroenterology: New Insights from Pig Models. Annu Rev Anim Biosci 2020; 8:321-354). Total PN is connected to shorter villi and crypts, more goblet cells, increased inflammation and immune cells, increased intercellular permeability and reduced blood flow.

As further described in Example 3.2, Villus height, midvillus width, and crypt depth were accordingly measured so as to understand the influence of various short-chain fatty acids, including arginine butyrate, on the integrity and functionality of the intestine.

It is known also that there is a relationship between intestinal epithelial integrity and intestinal health (Thomson et al., The Ussing chamber system for measuring intestinal permeability in health and disease, BMC Gastroenterology 2019; 19:98). Impairment of barrier function has been linked to intestinal diseases such as, for example, ulcerative colitis and Crohn's disease. The so-called Ussing system offers an ex vivo measurement of the permeability or duodenal mucosal resistance. The system allows to measure said duodenal mucosal (transepithelial) resistance (TER), which can be determined to give an overall measurement of gut integrity. A low TER value is indicative of increased permeability. Prior studies have shown that decreased TER under inflammatory conditions was associated with down regulation of “sealing” tight junctional proteins. Accordingly, determining epithelial integrity by means of the Ussing chamber constituted one option to assess the influence of various nutritional options (e.g. EN vs PN) and various PN compositions (see Example 3.5) on gut health and specifically on local inflammatory incidents.

Data in the context of the present invention have been obtained from a study with pigs. Pigs have become increasingly important animals for modeling human pediatric nutrition and gastroenterology and complementing mechanistic studies in rodents. The comparative advantages in size and physiology of the neonatal pig have led to new translational and clinically relevant models of important diseases of the gastrointestinal tract and liver in premature infants (Burrin et al, Annu Rev Anim Biosci 2020; 8:321-354). Therefore, comparative data to assess efficacy of different SCFA, specifically of arginine butyrate, as well as of SCFA-PN against standard PN and normal uptake of nutrition on gut barrier property, local and systemic inflammation and immunity as well as gut cellular architecture have been obtained based on a pig model (non-resected model, neonate pigs). Experiments were performed as described further in Example 1.

In said study for evaluating arginine butyrate in comparison to other butyric acid derivatives, specifically tributyrin and dipalmitoyl 3-butyryl glycerol, the effects on gut barrier functionality and related local and systemic effects on inflammation and immunity were determined (see Examples). Table 2 summarizes the high-level results of the Study, which show that all Intervention Groups (Groups which received PN with butyric acid derivative supplemented formulations) showed better results in terms of gut architecture, systemic and local inflammation and systemic and local immunity than the Group which obtained standard PN. Some of these effects have been described before in similar studies which mostly focused on the effect of butyrate provided as sodium butyrate or tributyrin. Surprisingly, a significant difference was found for formulations comprising arginine butyrate, which so far has not been used in parenteral nutrition compositions. Arginine butyrate proved to be especially beneficial regarding gut architecture, as evidenced by villus height, crypt depth and tight junctions (duodena mucosal resistance) analysis; local and systemic inflammation as shown by determination of pro-inflammatory and anti-inflammatory cytokines, and an improved local immunity as evidenced by sIgA. No difference for cognitive effects or brain development could be found for the respective intervention Groups.

TABLE 2
Summary of effects found in the Intervention Groups
receiving PN with different butyric acid derivatives.

| | | | | Group D |
|---|---|---|---|---|
| | | | Group C | (Dipalmitoyl |
| | Group A | Group B | (Arginine | butyryl |
| | (Tributyrin | (Tributyrin | Butyrate 10 | glycerol 10 |
| | 10 mmol/L) | 30 mmol/L) | mmol/L) | mmol/L |
| Tight | − | − | ++ | − |
| junctions | | | | |
| Villus | + | ++ | + | + |
| (total) | | | | |
| Crypt | + | ++ | + | + |
| (total) | | | | |
| Pro- | − | + | ++ | + |
| inflammatory | | | | |
| cytokines | | | | |
| Anti- | − | + | ++ | + |
| inflammatory | | | | |
| cytokines | | | | |
| IgA | ++ | ++ | +++ | ++ |
| Cognitive | − | − | − | − |
| Brain | − | − | − | − |

+ denotes a positive effect (compared to standard PN),
++ a very positive effect.
− denotes no difference.
Results are reported for villus height and crypt depth as “total”, which covers results for all sections reviewed (duodenum, jejunum, ileum, colon).

An immediate high-level comparison of the results in Intervention Group C, that received arginine butyrate, and the results intervention groups A and B, that received tributyrin which is known as potentially having positive effects on gut health, versus standard PN and enteral feeding (EN) supports the finding that arginine butyrate containing formulations have an unexpected superior effect on the tested markers for gut barrier functionality, local inflammation, local immunity, gut architecture and systemic inflammation (Table 3).

TABLE 3
High-level comparison between the effects of arginine
butyrates and tributyrin on selected markers for gut barrier
functionality, local inflammation, local immunity, gut architecture
and systemic inflammation.

| | | Arginine | Tributyrin¹ | |
|---|---|---|---|---|
| | | Butyrate¹ | 10 | 30 |
| Relevance | Marker/Test | (10 mmol/L) | mmol/L | mmol/L |
| Gut barrier | Duodenal Mucosal | ++² | = | = |
| functionality | Resistance (Ussing | | | |
| | chamber) | | | |
| Local | Jejunal Cytokine | +++ | ++ | ++ |
| inflammation | Concentration | | | |
| | (less pro- | | | |
| | inflammatory | | | |
| | cytokines I1-1β, I1-6, | | | |
| | TNF-α | | | |
| | Jejunal Cytokine | ++ | = | + |
| | Concentration | | | |
| | (more anti- | | | |
| | inflammatory | | | |
| | cytokines I1-8, I1-10) | | | |
| | Ileal Cytokine | ++ | = | + |
| | Concentration | | | |
| | (less pro- | | | |
| | inflammatory | | | |
| | cytokines I1-1β, I1-6, | | | |
| | TNF-α | | | |
| | Ileal Cytokine | +⁴ | − | + |
| | Concentration | | | |
| | (more anti- | | | |
| | inflammatory | | | |
| | cytokines I1-8, I1-10) | | | |
| Local | Jejunal sIgA | +++³ | ++⁵ | ++⁵ |
| immunity | Concentration | | | |
| Gut | Total villus | + | + | + |
| architecture | height | | | |
| | Total crypt depth | + | + | + |

+++ indicate P < 0.01;
++ indicates P < 0.05;
+ indicates positive trends;
= denotes no significant P value;
− denotes negative trends.
¹compared to standard PN (s-PN).
²as good as enteral feeding;
³better than enteral;
⁴less pro-inflammatory cytokines, downregulation of anti-inflammatory secretion;
⁵close to enteral;
⁶same trends as for local inflammation, to be further confirmed.

Accordingly, in pediatric patients, particularly in full-term neonates and pre-term infants, the composition according to the present disclosure can be used to support the development of a healthy intestinal morphology and/or growth and/or body composition and treat patients who have developed or at risk of developing a gastrointestinal disorder according to the invention. Furthermore, they can be used to improve immune response and gut flora. They are also useful for the resolution of intestinal inflammation including improving nutrient utilization. Specifically, they can be used in the treatment of short or extreme short bowel syndrome in pediatric patients.

While the compositions according to the present disclosure are particularly useful in treating infants, specifically pre-term neonates, they may equally well be used to treat adults suffering from same or similar conditions associated with gastrointestinal disorders as described herein.

The compositions according to the invention specifically support adult patients' local and systemic immune response, gut flora, and reduce local inflammation. Accordingly, they can be used for the treatment or resolution of inflammation and to improve nutrient utilization in patients being at risk of or who have already developed inflammation. Further, they may be used in the prevention or treatment of sepsis, chronic lung disease, cachexia, or inflammatory diseases. For example, the formulations according to the invention may be used in the treatment or prevention of cachexia and/or reduced immune response in cancer patients, of sepsis in critically ill patients, of metabolically stressed patients, or of parenteral nutrition associated issues in patients with short bowel syndrome or intestinal failure. They may also be used to support immune response in critically ill patients, to support immune response in cancer patients, to support immune response in immunodeficient patients, to support gut flora in metabolically stressed patients and to improve nutrient utilization in malnourished patients.

It is known (WO 2019/0232054 A1, WO2019/232044 A1) that lipid emulsions comprising choline derivatives have the potential to avoid and/or treat hepatic steatosis which may lead to liver metabolic dysfunction, inflammation, and advanced forms of nonalcoholic fatty liver disease (NAFLD) and which is an issue specifically in parenteral nutrition, especially in the treatment of pediatric patients but also in adult patients. NAFLD includes a spectrum of disease from simple steatosis to nonalcoholic steatohepatitis (NASH), which can progress to cirrhosis and hepatocellular carcinoma. Accordingly, the composition according to the invention may additionally contain choline derivatives, preferably choline chloride or GPC, which can address two, potentially interconnected, major issues arising in the total parenteral nutrition of patients, specifically of pediatric patients. Therefore, the present disclosure is also providing methods for treating hepatic steatosis, liver metabolic dysfunction, inflammation, and advanced forms of nonalcoholic fatty liver disease (NAFLD) in pediatric and/or adult patients, especially when occurring in concomitance with a gastrointestinal disorder according to the invention, sepsis, chronic lung disease, cachexia, or inflammatory diseases. Specifically, the compositions of the invention can be used to treat long-term TPN patients who have developed or are at risk of developing both hepatic steatosis, liver metabolic dysfunction, inflammation, and advanced forms of nonalcoholic fatty liver disease (NAFLD), a reduced gut barrier, degradation of the gut architecture, are suffering from incidents of chronic or acute inflammation (local and systemic) and have developed or at risk of developing a reduced local and systemic immunity.

The formulations according to the invention can be administered according to methods known in the art and as further disclosed herein.

### EXAMPLES

### Example 1: Materials and Methods

A pig model was chosen to assess the effects of enteral, standard parenteral, and parenteral nutrition wherein the standard PN formulation was supplemented with various butyrate derivatives (see Table 4). It is currently estimated that preterm pigs born at 90% gestation are comparable to human preterm infants at 75% gestation (30-32 weeks) (Burrin et al., Translational Advances in Pediatric Nutrition and Gastroenterology: New Insights from Pig Models. Annu Rev Anim Biosci 2020; 8:321-354).

### 1.1 Study Design

Neonatal Yorkshire/Landrace cross bred piglets (n=72; six at a time from the same litter, repeating 12 times) were obtained from Oak Hill Genetics (Ewing, Ill.) after 48-hour sow reared for colostrum consumption and iron supplementation. Piglets were randomized into Groups (12 piglets per group) to receive 10 days of nutrition as shown in Table 4.

TABLE 4
Nutrition provided to groups of piglets over 10 days.

| Group | Nutrition | Description |
|---|---|---|
| E | Milk replacer feeding taken ad | “Normal nutrition” |
| | libidum | |
| P | Olimel N9E | “Standard PN” |
| A | Olimel N9E + Tributyrin 10 mmol/L | Tributyrin provided |
| | TPN | in lipid chamber; |
| | | “TB-PN” |
| B | Olimel N9E + Tributyrin 30 mmol/L | Tributyrin provided |
| | TPN | in lipid chamber; |
| | | “TB-PN” |
| C | Olimel N9E + Arginine butyrate 10 | Arginine butyrate |
| | mmol/L TPN | provided in amino |
| | | acid chamber “AB-PN” |
| D | Olimel N9E + 1,2-Dipalmitoyl 3- | 1,2-Dipalmitoyl 3- |
| | butyryl glycerol 10 mmol/L TPN | butyryl glycerol |
| | | provided in lipid |
| | | chamber (DPBG-PN) |

The formulations used were all based on Olimel N9E (see Table 4). Supplementation with tributyrin and 1,2-dipalmitoyl 3-butyryl glycerol was provided in the lipid chambers of the 3CB product Olimel N9E, whereas arginine butyrate was added to the amino acid chamber. All concentrations given relate to the final reconstituted solution (TPN). Accordingly, the administered formulations and concentration are fully comparable, irrespective of the initial chamber to which the supplementation was added. To each bag of Olimel N9E that was administered, one bulk package Infuvite Pediatric (Baxter Healthcare Corp.) comprising vitamins for intravenous infusion after dilution was added. Also added to each bag of Olimel N9E was one vial MICRO +6 Pediatric Injection (Baxter Healtchare Corp.) comprising trace elements.

TABLE 5
Compositions used in the study.

| | Formulation | Formulation | Formulation | Formulation |
|---|---|---|---|---|
| Composition | Group A | Group B | Group C | Group D |
| Butyrate | Tributyrin | Tributyrin | Arginine | 1,2- |
| Derivative | (10 mmol/L | (30 mmol/L | butyrate | Dipalmitoyl |
| | TPN) | TPN) | (10 mmol/L | 3-butyryl |
| | (5 g/L LE) | (15 g/L LE) | TPN) | glycerol |
| | | | (6.6 g/L | (10 mmol/L |
| | | | amino | TPN) |
| | | | acids) | (32 g/L LE) |
| Amino Acid | 14.2 % | 14.2% | 14.5% | 14.2% |
| Chamber | | | | |
| concentration | | | | |
| Carbohydrate | 27.5% | 27.5% | 27.5% | 27.5% |
| Chamber | | | | |
| concentration | | | | |
| Lipid Chamber | 20.5% | 21.5% | 20.0% | 23.2% |
| concentration | | | | |
| Total Kcal | 1037 kCal/L | 1055 kCal/L | 1031 Kcal/L | 1085 Kcal/L |
| per L TPN | | | | |

LE means Lipid Emulsion.
Underlined values indicate which component of Olimel N9E was changed due to the addition of the supplement.
Olimel N9E alone: Amino Acid Chamber: 14.2%;
Carbohydrate Chamber 27.5%;
Lipid Emulsion Chamber 20.0%

Butyrate concentrations were chosen in a range which was known to be tolerated well in a similarly structured study (Bartholome et al, J Parent Nutr 2014; 28(4):210-223). Formulations were provided to achieve a daily amino acid content of 12.9 g/kg/day, with a daily infusion rate of 253 Kcal/kg/day. Only on day 1, immediately following surgery in order to minimize risk of malnutrition, the infusion rate was 307 Kcal/kg/day. A study period of 10 days was chosen due to the typical intestinal epithelial cell turnover time of 5-7 days in order to capture a complete cell turnover cycle.

### 1.2 Surgical Procedure

Upon arrival (Day 1), piglets underwent central line placement. A 3 cm incision was made in the right clavicular region to isolate the external jugular for catheter insertion (3.5 French polyvinyl chloride catheter). After blunt dissection, the jugular vein was ligated with two 3-0 silk sutures placed cranial (anatomically closer to head) and cardial (anatomically closer to heart) to the central line insertion site. Once the cranial ligature had been tied, a small incision was made in the jugular vein to insert a pre-measured central line (3.5 French PVC catheter) and was inserted 6 cm (premeasured and marked at 6 cm) through the external jugular in the super vena cava for PN infusion. Following central line placement, cardial suture was tied in order to immobilize the line, and the terminal end was tunneled subcutaneously to exit between the scapulae. Once placed, the central line was flushed with heparinized saline until attached to the PN pump. The incision site was closed in a single-layer closure using vicryl in a running subcuticular suture pattern. Suture sites were monitored and covered with petroleum jelly overlayed with sterile gauze anchored with Transpore tape.

### 1.3 Animal Care and Housing

Animals were allowed to recover under constant supervision and monitored for respiration rate, heart rate, signs of pain, and ensure regaining of consciousness. Following recovery, piglets will be fitted with jackets with the swivel tether (Lomir Biomedical Inc., Quebec, Canada) attached to protect the catheter and infusion lines to allow for free mobility. No pre-surgical jacket acclamation was done due to the young age and need to minimize time without nutrition and hydration. PN was be administered to provide 307 Kcal/kg/day immediately following surgery in order to minimize risk of malnutrition. The dosage of PN was 120% (20% higher) than the nutrition requirements for piglets of this age and size (200 Kcal/kg/day) to compensate for surgical stress. Each day, the animals underwent clinical assessment for both research and animal health purposes. A full clinical assessment was be performed every morning: weight (grams), girth (cm), body temperature, respiration rate, heart rate, activity level, healing of catheter insertion site and animal behavior and pain scores. A partial clinical assessment (minus weight and girth measurements) was performed each evening to reevaluate the wellness of the piglets.

### 1.4 Nutrient Interventions

Milk replacer (OptiLac Baby Pig Milk Replacer; Hubbard Feeds, Mankato, Minn., U.S.A.) were prepared fresh daily per manufacturer recommendation. Volume of milk replacer was calculated based on daily morning weight to provide 253 kcal/kg. The prepared volume of milk replacer was provided to Group E to be taken ad libitum.

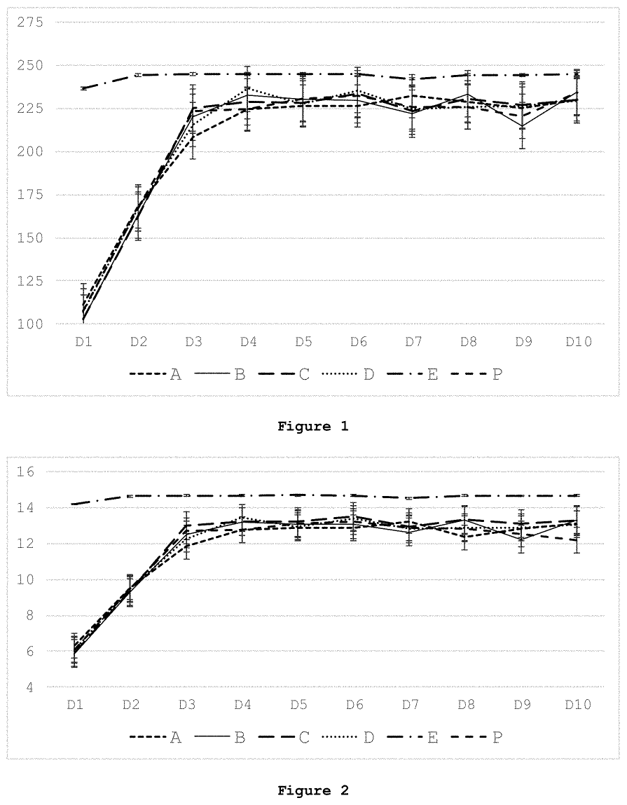

All PN solutions were compounded by the manufacturer (Baxter Healthcare Corp.) as a 3-in-1 solution (bag volume: 1000 mL) and delivered at the start of each experiment cycle and kept at 40° C. during administration. Each PN solution contained dextrose, amino acids, and a lipid emulsion in separate compartments until use. PN solutions also contained vitamins, minerals, and experimental amounts of butyric acid derivatives as assigned (Table 4 and 5). All PN solutions were infused continuously using AVA 6000CMS MultiTherapy infusion pumps (AVA Biomedical, Wilmette, Ill.) to provide 253 kcal/kg/day and 12.8 grams amino acids/kg/day. All milk replacer feedings and PN infusions were provided to ensure isocaloric provision for all piglets. Average energy delivery (FIG. 1) and average protein delivery (FIG. 2) were documented. The Figures demonstrate that the Study Groups were investigated under the same conditions.

### 1.5 Cognitive Assessment

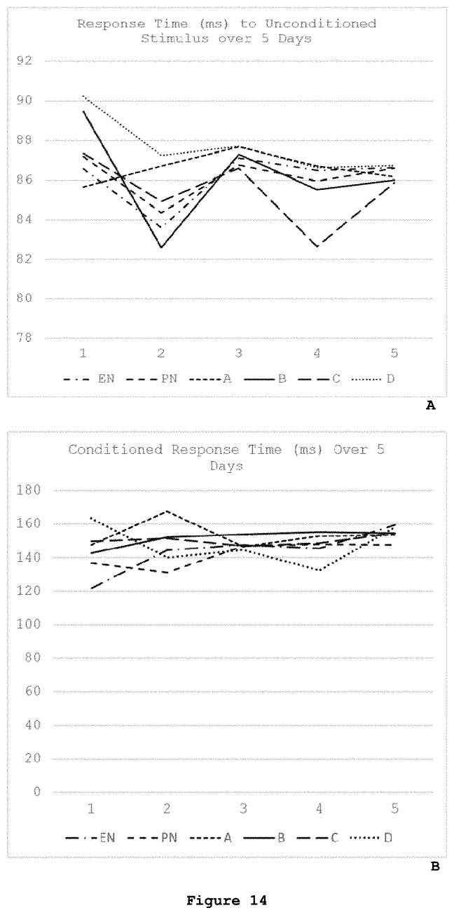

Eyeblink conditioning is an established Pavlovian method to assess the cerebellum and associated brainstem circuitry with hippocampus involvement that are essential for learning and memory. The eyeblink conditioning procedure took place in a sound-attenuating chamber. A fan was inside the chamber and ran throughout the experiment for ambient noise (70 dB). A speaker mounted to the wall of the chamber delivered the tone conditioned stimulus (CS). A small plastic air puff delivery nozzle (San Diego Instruments, San Diego, Calif.) was secured at approximately 2 cm from the piglet's left eye to deliver the unconditioned stimulus (US). After adaptation to the conditioning apparatus on study day 3, a total of five CS-US conditioning sessions occurred on study days 4-8 following. Each conditioning session consisted of 90 CS-US paired trials and 10 CS alone trials for a total of 100 trials/session. Every tenth trial was a CS alone trial. The CS-US paired trials included: a 500 ms auditory CS (1 kHz, 85 dB tone), a 400 ms interstimulus interval (ISI), followed by a 100 ms corneal airpuff US (10 psi). Both the CS and US were co-terminated at the exact same time. The CS alone trials consisted of a 500 ms auditory CS (1 kHz, 85 dB tone) only. There were random inter-trial intervals of 20 seconds throughout each session. Each conditioning session lasted no longer than 35 minutes. The San Diego Instruments eyeblink software was used to record conditioning session moment-to-moment infrared-reflectance data. The results are shown in FIG. 14. No significant differences were detected between the Groups.

### 1.6 Brain Structure Development and Body Composition Assessment

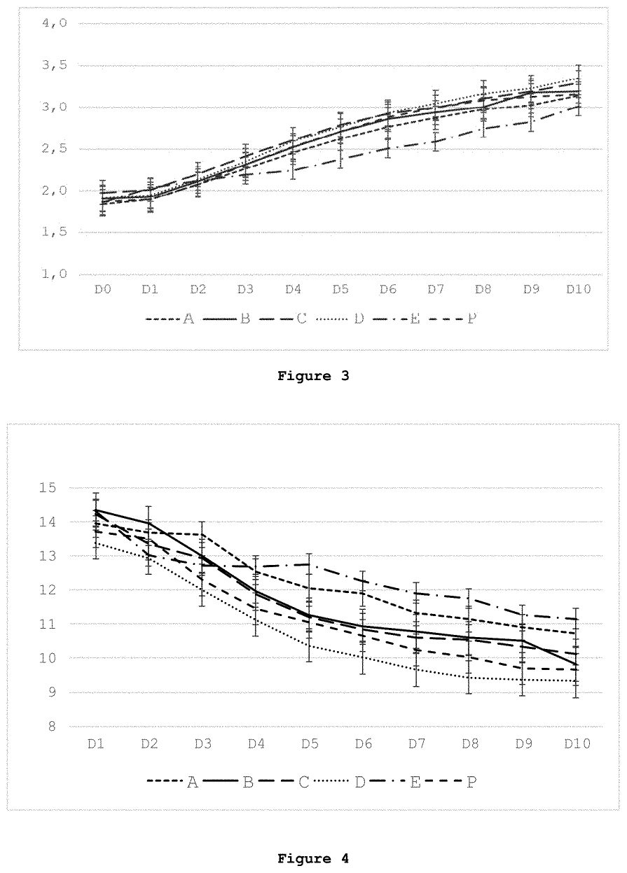

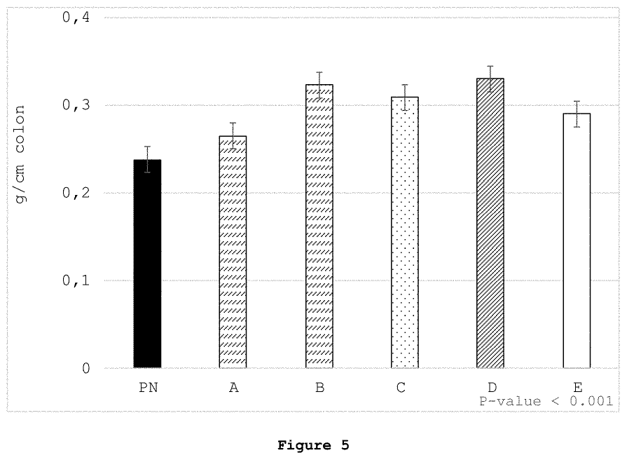

On study day 9, the piglets were submitted to Magnetic Resonance Imaging (MRI) using an Agilent 9.4 Tesla MRI system (Santa Clara, Calif.) to assess brain structural development and body composition. Heart rate and respiration were monitored during the entire MRI scan. For anatomic assessment of the brain targeting the hippocampus, images were obtained using a 3D T1-weighted magnetization prepared gradient-echo sequence: repetition time=1,900 ms; echo time=2.48 ms; inversion time=900 ms, flip angle=9°, matrix=256×256 (interpolated to 512×512), slice thickness=1.0 mm. For body composition assessment, a multi-slice, spin-echo technique was used to image the truncal area (longissimus fat/muscle): echo time=20 ms; recovery time=400 ms with four-signal averaging. Each image had a slice thickness of 4.9 mm with no gap between images. Total imaging time did not exceed one hour per piglet. Total scanning time was approximately 60 minutes to include both brain and body composition scanning. The MRI images were analyzed using OsiriX (Bernex, Switzerland). It was found that the brain structure did not show any significant differences between the Study Groups. The average body weight and abdominal girth of the piglets in the Study Groups was also monitored. FIG. 3 shows that the body weight developed similarly in all Study Groups. FIG. 4 shows that also the average abdominal girth developed similarly in the Study Groups.

### 1.7 Study Sample Collection

Upon completion of the study period, animals were euthanized by lethal injection (1 mL/10 lb; Fatal Plus; Veterinary Laboratories, Inc, Lenexa, Kans.) delivered via the central line. Urine and blood samples were collected for chemistry, high-performance liquid chromatography (HPLC), and enzyme-linked immunosorbent assay (ELISA) measurements. The gastrointestinal tract was removed and separated into different anatomic segments for histomorphology, electrophysiology, nutrient transport, and ELISA tests. Kidneys, liver, spleen, and muscle samples were also taken from each piglet for histology and chemistry assessments. Stool samples were collected and stored for microbiota analyses. The average colonic weight of the Study Groups was determined (FIG. 5). Here, the Groups A to D which had obtained butyric acid derivative supplemented PN and the Group that had received enteral feeding were superior to Group PN that has obtained standard PN.

### Example 2: Compositions Tested

Compositions used are described in Table 4 and Table 5. After compounding of the respective butyrate derivatives into the respective chambers of the bags and following sterilization, free butyric acid was measured in the lipid chamber (for tributyrin and DPBG compositions) and the butyrate content in the amino acid chamber was determined. As shown in Table 5, only very limited amounts of butyric acid were released during the sterilization of the bags. In case of the amino acid chamber, the recovery of butyrate after sterilization corresponds to what was introduced. Accordingly, no deterioration of the salt occurred during or after sterilization. In addition, the stability of the composition over time (12 months, 25° C., 40% RH) was confirmed. The free butyric acid can be quantified by GC-FID, after sample preparation by liquid-liquid extraction of the lipid emulsion or directly from amino acid solution. These methods are known in the art.

### Example 3: Methods for Assessing Study Endpoints

### 3.1 Fisher's Least Significant Difference (LSD) Test

The method of Fisher's Least Significant Difference (LSD) Test has been described, for example, by Williams and Abdi in Neil Salkind (Ed.), Encyclopedia of Research Design. Thousand Oaks, Calif.: Sage. 2010. The Fisher's LSD test is basically a set of individual tests. It is only used as a follow up to ANOVA. Following one-way (or two-way) analysis of variance (ANOVA), it is possible to compare the mean of one group with the mean of another. One way to do this is by using Fisher's Least Significant Difference (LSD) test. The test follows the principle to compute the smallest significant difference (i.e., the LSD) between two means as if these means had been the only means to be compared (i.e., with a t test) and to declare significant any difference larger than the LSD.

### 3.2 Histomorphology and Preparation of Ileus and Jejunum Sections

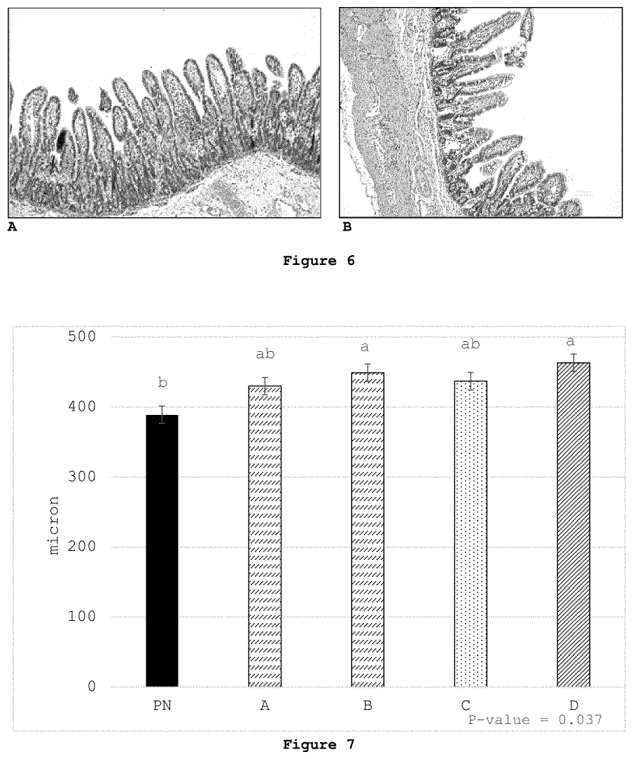

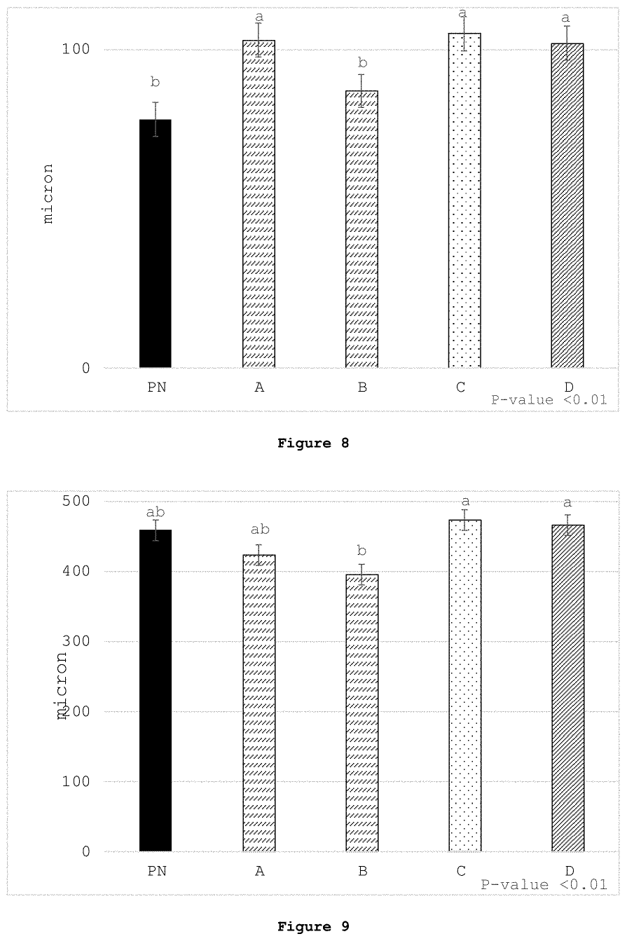

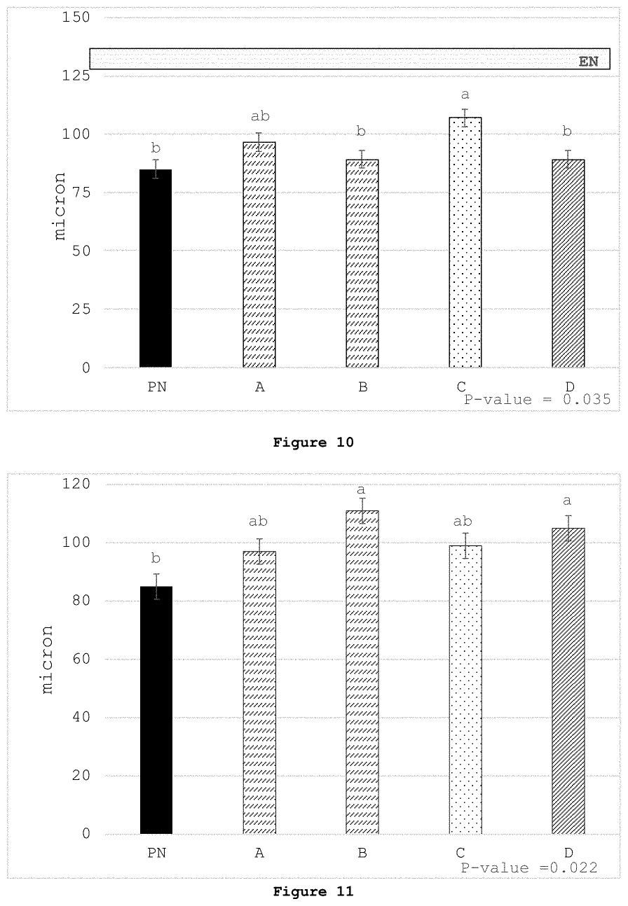

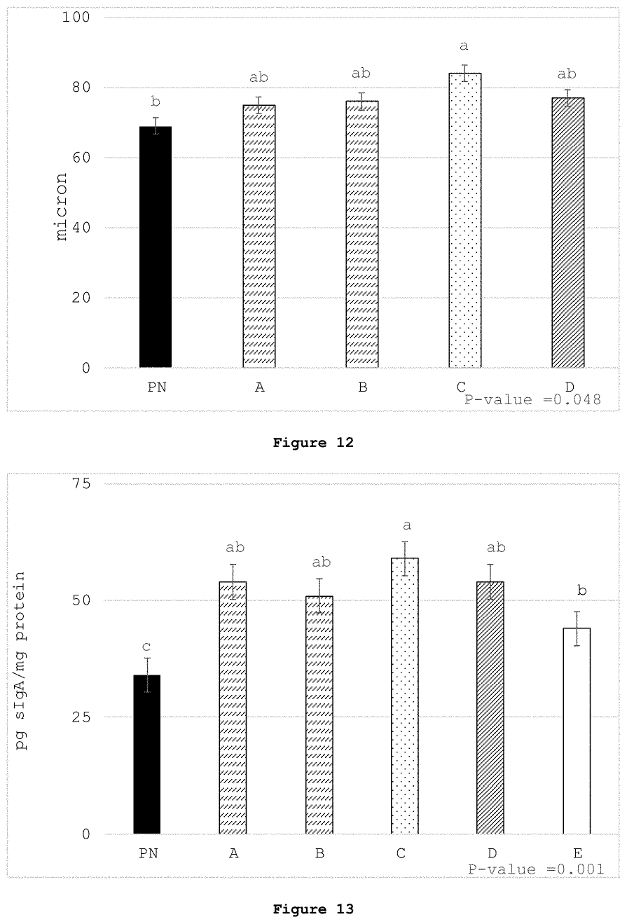

Formalin-fixed intestinal samples were embedded in paraffin, sliced to approximately 5-μm thickness with a microtome and stained with hematoxylin and eosin (FIG. 6). Villus height, midvillus width, and crypt depth were measured by using a Nikon Optiphot-2 microscope (Nikon, Melville, N.Y.) and ImagePro Express software (Version 4.5; Media Cybernetics, Inc, Silver Spring, Md.) in 8 to 10 well-oriented villi and crypts. Villus surface area (villus height×midvillus width) was also calculated. In addition, intestinal segment circumference was measured to estimate intestinal surface area. The results are provided in FIGS. 7 through 12 (see there for details). Table 6 shows the results of the histomorphological analysis of the Study Groups. The Table summarizes the average difference (in % of each Intervention Group (A, B, C, D) versus the PN Group) of the villus height and the crypt depth in the different sections of the gut, i.e. duodenum, jejunum, ileum and colon. The Intervention Groups show an improved villus height compared to the s-PN Group. In total Group C shows the most prominent improvement of the gut architecture.

TABLE 6
Histomorphological Results of the Intervention Groups, providing for the average difference
(in % of each Intervention Group (A, B, C, D) versus the PN Group) of the villus height
and the crypt depth in the different sections of the gut.

| % Δ | | | | | | | | | | |
|---|---|---|---|---|---|---|---|---|---|---|
| from | Duodenum | | Jejunum | | Ileum | | Colon | Total | | |
| PN | C | V | C | V | C | V | C | C | V | Total |
| A | +11 | +32 | −8 | +14 | +17 | +14 | +9 | +29 | +60 | +89 |
| B | +12 | +12 | −14 | +5 | +27 | +31 | +10 | +63 | +48 | +111 |
| C | +15 | +35 | +3 | +26 | +7 | +16 | +22 | +47 | +77 | +124 |
| D | +19 | +31 | +2 | +5 | +15 | +24 | +12 | +50 | +60 | +110 |

### 3.3 Plasma Glucagon Like Peptide 2 (GLP-2) Concentrations

Plasma GLP-2 concentration is quantified by extracting plasma samples with 75% ethanol and centrifugation at 3000×g for 30 minutes at 4° C. The supernatant was decanted, lyophilized, and reconstituted to the original plasma volume in assay buffer (80 mmol/L Na₃PO₄⁻, 0.01 mmol/L valine-pyrrolidine, 0.1% wt/vol human serum albumin, 10 mmol/L EDTA, 0.6 mmol/L thimerosal, pH 7.5). Approximately 300 μL of extracted samples and human GLP-2 standards are incubated with 100 μL of rabbit GLP-2 antiserum (final dilution 1:25,000) for 24 hours at 4° C., after which free and bound peptides are separated by absorption to plasma-coated charcoal (see also Bartholome et al, J Parent Nutr 2004; 28(4):210-223 for standards used). This antiserum is raised against the NH₂-terminal fragment of human GLP-2 and specifically recognizes the NH₂-terminal region of both human and porcine GLP-2.

### 3.4 IgA Quantification

Small intestine probes for IgA level determination were obtained by flushing the small intestine with chilled HBSS (Hank's Balanced Salt Solution). Nasal and bronchoalveolar washings for measurement of respiratory tract IgA levels were obtained by lavage with 1 mL phosphate-buffered saline solution under anesthesia. The washings were stored in a −80° C. freezer until IgA analysis. IgA was measured in small intestinal and respiratory tract washings by sandwich enzyme-linked immunosorbent assay using a polyclonal goat anti-mouse IgA (Sigma) to coat the plate, a purified mouse IgA (Zymed Laboratories, San Francisco, Calif.) as the standard, and a horseradish peroxidase-conjugated goat anti-mouse IgA (Sigma). Results are shown in FIG. 13.

### 3.5 Duodenal Mucosal Resistance

Duodenal mucosal resistance was determined according to known methods and according to Tappenden et al., Short-Chain Fatty Acid-Supplemented Total Parenteral Nutrition Enhances Functional Adaptation to Intestinal Resection in Rats. Gastroenterology 1997; 112:792-802. Pieces of intestine (1 cm²) were cut out, and the tissue was mounted as flat sheets in incubation chambers containing oxygenated Krebs' bicarbonate buffer (pH 7.4) at 37° C. Tissue discs were preincubated in this buffer for 15 minutes to allow equilibration at this temperature. After preincubation, the chambers were transferred to other beakers containing [³H]inulin and various 14C probe molecules in oxygenated Krebs' bicarbonate (pH 7.4 and 37° C.). The concentration of solutes was 4, 8, 16, 32, or 64 mmol/L for D-glucose and 16 mmol/L for L-glucose. The preincubation and incubation solutions were mixed at identical stirring rates with circular magnetic bars, and the stirring rates were adjusted by means of a strobe light. A stirring rate of 600 rpm was selected to achieve low effective resistance of the intestinal unstirred water. The experiment was terminated by removing the chambers and quickly rinsing the tissue in cold saline for approximately 5 seconds. The exposed mucosal tissue was then cut out of the chamber with a circular steel punch. For all probes, the tissue was dried overnight in an oven at 55° C. The dry weight of the tissue was determined, the sample was saponified with 0.75N NaOH, scintillation fluid was added (Beckman Ready Solv HP; Beckman, Mississauga, ON), and radioactivity was determined by means of volume of an external standardization technique to correct for variable quenching of the two isotopes. The mucosal weight was determined after scraping of the intestine from adjacent samples not used for uptake studies. The weight of the mucosa in the samples used to measure uptake was determined by multiplying the dry weight of the intestinal sample by the percentage of the intestinal wall comprised of mucosa. Results of the measurements for all Groups are provided in Table 7.

It was found that the Intervention Groups A, B, C and D, are significantly different from the PN (parenteral nutrition) reference group (see Table 2) as regards duodenal mucosal resistance. Moreover, all interventions groups were not inferior or statistically different from Group EN (enteral nutrition). Intervention Group D resulted in duodenal resistance which is almost as good as in Group E (enteral). Remarkably, Group C (arginine butyrate) showed a significantly lower loss of mucosal resistance compared to the other intervention groups, and is even better than the enteral Group E, indicating that the addition of arginine butyrate is surprisingly effective in maintaining or supporting duodenal mucosal resistance and improved gut health, including a low susceptibility for local inflammation.

TABLE 7
Mean duodenal mucosal resistance in the respective
intervention Groups A-D, compared with S-PN (standard PN) and EN
(enteral nutrition), as determined by Fisher's Least Significant
Difference (LSD) Test. Group C (AB-PN) shows a significantly
better resistance in comparison to other intervention groups and
enteral nutrition Group E.

| Group | | | |
|---|---|---|---|
| Assignment | N | Mean | Grouping |
| PN | 12 | 34.2533 | a |
| A | 12 | 21.8442 | ab |
| B | 12 | 16.0234 | ab |
| D | 12 | 15.8264 | ab |
| EN | 13 | 15.2746 | b |
| C | 12 | 8.2593 | b |

### 3.6 Cytokine Quantification

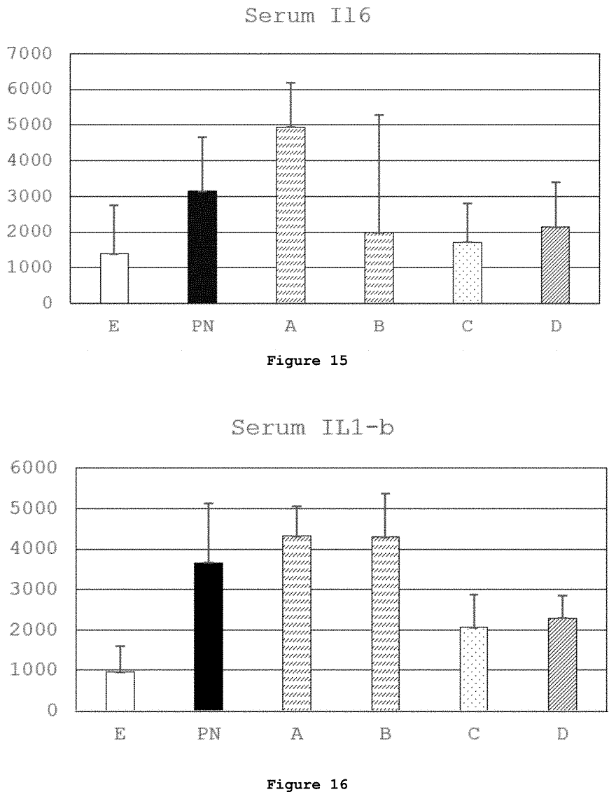

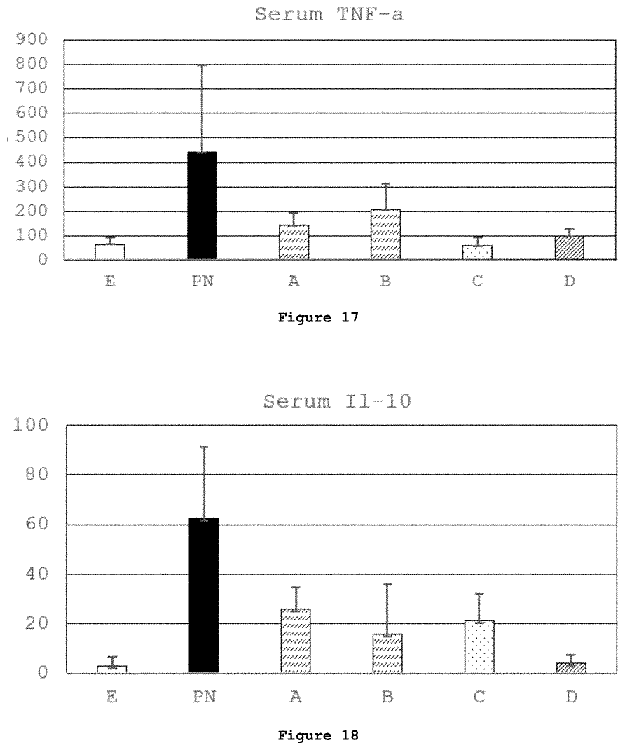

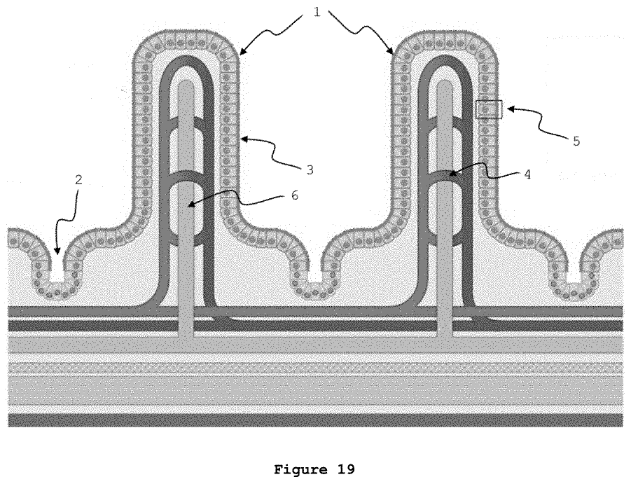

The influence of the respective supplements on inflammation was investigated by determining Il-6, Il-1beta, TNF-alpha and Il-10 serum levels in the intervention groups compared to EN and PN according to Milo et al., Effects of Short-Chain Fatty Acid-Supplemented Total Parenteral Nutrition on Intestinal Pro-Inflammatory Cytokine Abundance. Digestive Diseases and Sciences 2002; 47:2049-2055. Jejunal and ileal samples were homogenized in double-distilled water, and the Bradford protein assay (Biorad, Hercules, Calif., USA) was performed on homogenate and plasma samples. Protein (30 μg) from each sample was denatured by boiling, and proteins were separated by size using 12.5% sodium dodecyl sulfate polyacrylamide gel electrophoresis. Separated proteins were transferred to polyvinylidene difluoride membranes (Biorad) using a semidry transfer apparatus (Biorad). Western blot analysis for TNF-alpha, IL-1beta, Il-10 and IL-6 was performed using porcine-specific polyclonal antibodies (Endogen, Woburn, Mass., USA). A mouse anti-pig monoclonal TNF-alpha antibody was used to detect TNF-alpha (17,000 kDa). Rabbit anti-pig polyclonal antibodies specific for IL-1beta and IL-6 were used to detect IL-1beta (17,500 kDa), Il-10 (18,600 kDa) and IL-6 (26,000 kDa). Membranes were developed using the Opti-4CN kit (Biorad) and photographed using the FOTO/Analyst Image Analysis System (Fotodyne, Inc., Hartland, Wis., USA). Densitometry of TNF-alpha, IL-1beta, IL-6, and Il-10 was performed using Collage Image Analysis Software 4.0 (Fotodyne, Inc.). Results are shown in FIG. 15 (Il-6), FIG. 16 (Illbeta), FIG. 17 (TNF-alpha) and FIG. 18 (Il-10).

### Example 4: Preparation of an Aqueous Composition of Arginine Butyrate

174.55 g arginine free base (purity 99.8%), (1 equivalent) were dissolved in 450 mL water (90% of the final volume) while cooling the solution to about 10° C. under vigorous stirring. Some precipitation could be observed. Then, 88.29 g of butyric acid (purity 99.8%) (1 equivalent) were added. Care was taken so the temperature in the reaction vessel would not exceed a temperature of 25° C. Finally, the remaining quantity of water was added (10% of the final volume) while further stirring the solution at room temperature. pH was checked, completion of the salt formation is considered done when pH reaches 7.5+/−0.5. Upon filtration through a 0.45 μfilter to remove any precipitated material, the solution was filled into multilayered bags. Bags were sterilized in a hot steam-air mixture (121° C., 2.2 bar). After sterilization, bags were overpouched and stored at room temperature until use.

### Example 5: Preparation of a Lyophilized Composition Comprising Arginine Butyrate

For arginine butyrate solution preparation see example 4. Upon neutralization and pH adjustment to a value of 7.2, glass vials are filled with the mother solution. The filled glass vials are then lyophilized (specific cycle to be applied according to the solution concentration and the volume of the vials). At the end of the lyophilization, vials are capped and stored either at cold temperature (5+/−2° C.) or at room temperature.

1. A composition for use in the treatment of a patient suffering from or being at risk of developing a gastrointestinal disorder, wherein the composition is an aqueous solution for injection comprising arginine butyrate in a concentration of from 150 mg/L to 5500 mg/L of the composition.

2. The composition of claim 1, wherein the composition is essentially free of amino acids or carbohydrates.

3. The composition of claim 1, wherein the composition further comprises one or more water-soluble vitamins selected from the group of vitamins consisting of vitamin C (ascorbic acid), vitamin B1 (thiamine), vitamin B2 (riboflavin), vitamin B3 (niacin), vitamin B5 (pantothenic acid), vitamin B6 (pyridoxine), vitamin B12 (cyanocobalamin), biotin and folic acid.

4. The composition according to claim 1, wherein the composition further comprises one or more trace elements selected from the group of trace elements consisting of copper, chromium, fluoride, iodine, iron, manganese, molybdenum, selenium and zinc.

5. The composition according to claim 1, wherein arginine butyrate is present in a concentration of from 250 g to 5.5 g per liter, from 260 mg to 4.5 g per liter, from 280 mg to 3.5 g per liter, from 300 mg to 2.5 g per liter, from 320 mg to 1.5 g per liter, or from 350 mg to 800 mg per liter.

6. The composition according to claim 1, wherein the pH of the composition is from 5.0 to 8.0.

7. The composition according to claim 1, wherein arginine butyrate is the sole active ingredient.

8. The composition according to claim 1, wherein the patient is a pediatric or an adult patient receiving parenteral nutrition.

9. The composition according to claim 1, wherein the patient suffers from or is at risk of developing intestinal inflammation, reduced local immunity in the gut, and/or reduced gut barrier functionality.

10. The composition according to claim 1, wherein the patient is any ICU patient, a critically ill patient, a home PN patient, or a hospitalized patient with a minimum enteral nutrition covering more than 50%, preferably more than 60%, 70%, 80% or 90% of the energy needs from parenteral nutrition.

11. The composition according to claim 1, wherein the patient is a short bowel patient, an extreme short bowel patient, an intestinal failure patient, a metabolically stressed patient, an immunodeficient patient, a cancer patient, a cachexia patient, a malnourished patient, a patient suffering from or being at risk of developing hyperglycemia and/or hypertriglyceridemia, a patient suffering from or being at risk of developing gut motility reduction, a patient with burns or trauma, a patient developing IFALD.

12. The composition according to claim 1, wherein the patient is a short bowel patient, an extreme short bowel patient or an intestinal failure patient.

13. The composition according to claim 1, wherein the patient suffers from or is at risk of developing intestinal failure.

14. The composition according to claim 1, wherein the patient suffers from or is at risk of developing short bowel syndrome.

15. The composition according to claim 1, wherein the composition is administered to the patient by intravenous, intramuscular or subcutaneous injection, preferably by intravenous injection.

16. The composition according to claim 1, wherein the composition is added to a parenteral nutrition formulation during compounding or is admixed with a pre-mixed parenteral nutrition formulation before administration to the patient.

17. The composition of claim 1, wherein the composition is administered to the patient to arrive at an arginine butyrate dose of from 5 mg/kg/day to 10 g/kg/day.

18. The composition of claim 1, wherein the composition is administered to the patient to arrive at an arginine butyrate dose of from 5 mg/kg/day to 5 g/kg/day.

19. A method of treating a patient suffering from or being at risk of developing a gastrointestinal disorder, comprising administering a pharmaceutically sufficient amount of the composition according to claim 1.

20. The method of claim 19, wherein the patient is a pediatric or an adult patient receiving parenteral nutrition.

21. The method of claim 19, wherein the patient is any ICU patient, a critically ill patient, a home PN patient, or a hospitalized patient with a minimum enteral nutrition covering more than 50%, preferably more than 60%, 70%, 80% or 90% of the energy needs from parenteral nutrition.

22. The method of claim 19, wherein the patient is a short bowel patient, an extreme short bowel patient, an intestinal failure patient, a metabolically stressed patient, an immunodeficient patient, a cancer patient, a cachexia patient, a malnourished patient, a patient suffering from or being at risk of developing hyperglycemia and/or hypertriglyceridemia, a patient suffering from or being at risk of developing gut motility reduction, a patient with burns or trauma, a patient developing IFALD.

23. The method of claim 19, wherein the patient is a short bowel patient, an extreme short bowel patient or an intestinal failure patient.

24. The method of claim 19, wherein the patient suffers from or is at risk of developing intestinal failure.

25. The method of claim 19, wherein the patient suffers from or is at risk of developing short bowel syndrome.

26. The method of claim 19, wherein the composition is administered to the patient to arrive at an arginine butyrate dose of from 5 mg/kg/day to 10 g/kg/day.

27. The method of claim 19, wherein the composition is administered to the patient to arrive at an arginine butyrate dose of from 5 mg/kg/day to 5 g/kg/day.
